(19) United States
(12) Patent Application Publication
(10) Pub. No.: US 2024/0101787 A1
(43) Pub. Date: Mar. 28, 2024

(54) CROSSLINKING AGENTS

(71) Applicant: The Board of Trustees of The University of Alabama, (US)

(72) Inventors: Jason Edward Bara, Tuscaloosa, AL (US); Amber Renee Bara, Tuscaloosa, AL (US); Shuai Qian, Northport, AL (US)

(21) Appl. No.: 18/238,684

(22) Filed: Aug. 28, 2023

Related U.S. Application Data
(60) Provisional application No. 63/401,236, filed on Aug. 26, 2022.

(51) Int. Cl. C08K 5/1565; A61K 8/85; A61Q 5/00; C08G 63/183; C08L 67/02

(52) U.S. Cl. CPC C08K 5/1565; A61K 8/85; A61Q 5/00; C08G 63/183; C08L 67/02; A61K 2800/95; C08L 2207/20

(57) ABSTRACT

The present disclosure describes crosslinking agents derived from glycerol and/or waste poly(ethylene terephthalate) of Formula I and Formula II which may find use in various polymer applications, including as an additive in personal care products.

### CROSS-REFERENCE TO RELATED APPLICATIONS

This application claims the benefit of priority to U.S. Provisional Application No. 63,401,236 filed Aug. 26, 2022, the disclosure of which is incorporated herein by reference in its entirety.

### STATEMENT REGARDING FEDERALLY SPONSORED RESEARCH OR DEVELOPMENT

This invention was made with government support under Grant Nos. 2029387 and 2132133, both awarded by the National Science Foundation. The Government has certain rights in the invention.

### TECHNICAL FIELD

This disclosure relates to crosslinking agents, and more particularly to crosslinking agents derived from glycerol or waste polyethylene terephthalate, which find use in various polymer applications, including as additives in personal care products.

### SUMMARY

The present disclosure provides crosslinking agents derived from glycerol or waste polyethylene terephthalate, which may find use in numerous applications, for example, from polymer upcycling to hair and skincare products.

In one aspect, a crosslinking agent is provided of Formula I

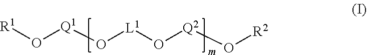

wherein all variables are as defined further herein.

In another aspect, a crosslinking agent is provided of Formula II

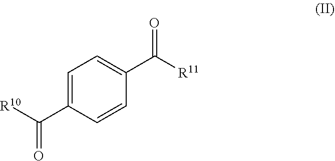

wherein all variables are as defined further herein.

In a further aspect, a polymer composition is provided comprising at least one polymer crosslinked with a crosslinking agent described herein. Articles formed from such polymer compositions are also provided.

In a further aspect, a resin composition comprising at least one monomer and a crosslinking agent described herein is also provided.

In another aspect, a personal care composition is provided comprising a crosslinking agent described herein. In some aspects, the personal care composition is a haircare product.

The details of one or more aspects of the disclosure are set forth in the description below. Other features, objects, and advantages of the disclosure will be apparent from the description and the claims.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the foregoing descriptions and the associated drawings. Therefore, it is to be understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

As can be apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to but does not preclude the presence or addition of one or more features, integers, steps, or components, or groups thereof. Moreover, each of the terms “by,” “comprising,” “comprises,” “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, the term “consisting essentially of” is intended to include examples encompassed by the term “consisting of.

As used in the specification and the appended claims, the singular forms “a,” “an,” and “the” include plural referents unless the context clearly dictates otherwise. Thus, for example, reference to “a polymer,” “an agent,” or “the composition” includes, but is not limited to, two or more such polymers, agents, or compositions, and the like.

It should be noted that ratios, concentrations, amounts, and other numerical data can be expressed herein in a range format. It can be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. It is understood that where “about,” “approximate,” or “at or about” is used before a quantitative value, the parameter also includes the specific quantitative value itself unless specifically stated otherwise.

As used herein, the terms “optional” or “optionally” mean that the subsequently described event or circumstance can or cannot occur and that the description includes instances where said event or circumstance occurs and instances where it does not.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to the administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., ═O), then two hydrogens on the atom are replaced. For example, a pyridyl group substituted by oxo is a pyridine. Combinations of substituents and/or variables are permissible only if such combinations result in stable compounds or useful synthetic intermediates. A stable compound refers to a compound that can be isolated and has a shelf life of at least one month. A stable manufacturing intermediate or precursor to a compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for various functional groups as used and defined herein are not intended to be limited to monovalent radicals and may include polyvalent radical groups as appropriate, such as divalent, trivalent, tetravalent, pentavalent, and hexavalent radicals, and the like, based on the position and location of such groups in the compounds described herein as would be readily understood by the skilled person.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges, as used herein, indicate an alkyl group with the length of each member of the range described as an independent species. For example, C₁-C₆alkyl, as used herein, indicates an alkyl group having from 1, 2, 3, 4, 5, or 6 carbon atoms and is intended to mean that each of these is described as an independent species, and C₁-C₄alkyl, as used herein, indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case, cycloalkyl, is either directly bound by a single covalent bond (C₀alkyl), or attached by an alkyl chain, in this case, 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one aspect, the alkyl group is optionally substituted as described herein.

“Cycloalkyl” is a saturated mono- or multicyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one aspect, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group, as defined above, covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group, as defined above, with the indicated number of carbon atoms covalently bound through a sulfur bridge (—S—). In one aspect, the alkoxy group is optionally substituted as described herein.

“Alkanoyl” is an alkyl group, as defined above, covalently bound through a carbonyl (C═O) bridge. The carbonyl carbon is included in the number of carbons. For example, C₂alkanoyl is a CH₃(C═O)— group. In one aspect, the alkanoyl group is optionally substituted as described herein.

“Halo” or “halogen” indicates, independently, any of fluoro, chloro, bromo, or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include the fusion to a 4- to 7- or 5- to 7-membered saturated or partially unsaturated cyclic group that optionally contains 1, 2, or 3 heteroatoms independently selected from N, O, B, P, Si, and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include but are not limited to dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3 -dihy dro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example, indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring that contains from 1 to 4, or in some aspects, 1, 2, or 3 heteroatoms selected from O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is, groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group exceeds 1, these heteroatoms are not adjacent to one another. In one aspect, the total number of S and O atoms in the heteroaryl group is not more than 2. In another aspect, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high-performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties of the substance. Both traditional and modern methods for purification of the compounds to produce substantially chemically pure compounds are known to those of skill in the art. A substantially chemically pure compound may, however, be a mixture of stereoisomers.

The present disclosure provides crosslinking agents derived from glycerol and/or waste poly(ethylene terephthalate), which may find use in such applications as the modification of other polymers, protein conjugation, personal care products, thermoset formulations, and additive manufacturing.

In one aspect, a crosslinking agent is provided of Formula I

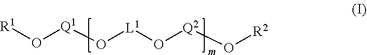

wherein:

m is 0 or 1;

Q¹ and Q² if present, are independently selected from:

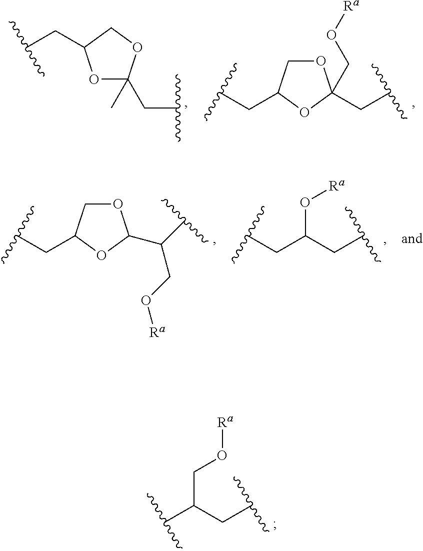

L¹ if present, is selected from C₁-C₁₀ alkyl, C₃-C₈ cycloalkyl, 3- to 8-membered monocyclic or bicyclic heterocycle, 6- to 10-membered monocyclic or bicyclic aryl, and 5-to 10-membered monocyclic or bicyclic heteroaryl, each of which may be optionally substituted with one or more X substituents as allowed by valency;

R^{a} is independently selected at each occurrence from hydrogen, R¹, C₁-C₁₀ to alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;

R¹ and R² are independently selected at each occurrence from

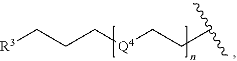

wherein n is selected at each occurrence from 0, 1, 2, or 3;

R³ is selected at each occurrence from

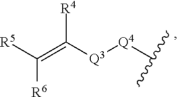

—NH₂, —NH₃⁺Z³¹ , and

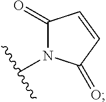 [SMILES: *N1C(=O)C=CC1=O]

Q³ is absent or independently selected at each occurrence from —C(═O)— and —S(═O)₂—;

Q⁴ is independently selected at each occurrence from —O— and —NH—;

R⁴ , R⁵ , and R⁶ are each independently selected at each occurrence from hydrogen, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;

Z⁻ is selected at each occurrence from a halide,

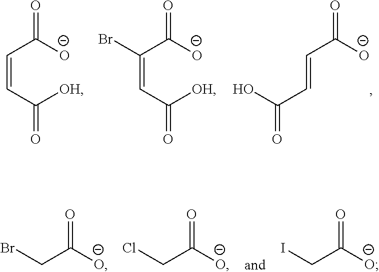

X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, to R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;

R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;

R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and

Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some aspects of Formula I, m is 0. In some aspects of Formula I, m is 1.

In some aspects of Formula I, Q² is

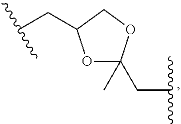 [SMILES: *CC1COC(C)(C*)O1]

In some aspects of Formula I, Q² is

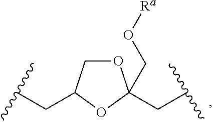

In some aspects of Formula I, Q² is

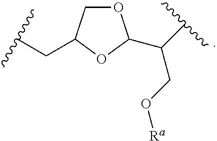

In some aspects of Formula I, Q² is

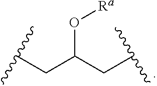

In some aspects of Formula I, Q² is

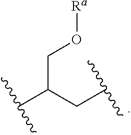

In some aspects of Formula I, L¹ is C₁-C₁₀ alkyl. In some aspects of Formula I, L¹ is selected from C₁ alkyl, C₂ alkyl, C₃ alkyl, C₄ alkyl, C₅ alkyl, C₆ alkyl, C₇ alkyl, C₈ alkyl, C₉ alkyl, and C₁₀ alkyl.

In some aspects of Formula I, Q¹ is

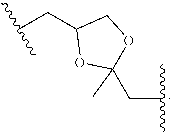 [SMILES: *CC1COC(C)(C*)O1]

In some aspects of Formula I, Q¹ is

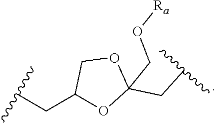

In some aspects of Formula I, Q¹ is

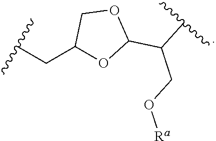

In some aspects of Formula I, Q¹ is

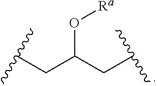

In some aspects of Formula I, Q ¹ is

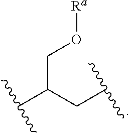

In some aspects of Formula I, R^{a} is selected at each occurrence from hydrogen, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl. In some aspects of Formula I, R^{a} is R¹.

In some aspects of Formula I, R¹ and R² are each

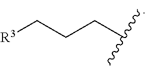

In some aspects of Formula I, R¹ and R² are each

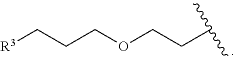

In some aspects of Formula I, R¹ and R² are each

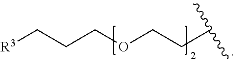

In some aspects of Formula I, R¹ and R² are each

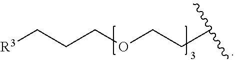

In some aspects of Formula I, R³ is

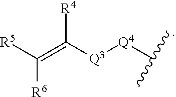

In some aspects of Formula I, Q³ is absent. In some aspects of Formula I, Q³ is —C(═O)—.

In some aspects of Formula I, Q⁴ is —O—. In some aspects of Formula I, Q⁴ is —NH—.

In some aspects, R⁴, R⁵, and R⁶ are each hydrogen. In some aspects, R⁵ is phenyl, and R⁴ and R⁶ are each hydrogen. In some aspects, R⁴ is methyl and R⁵ and R⁶ are each hydrogen.

In some aspects, R³ is —NH₂. In some aspects, R³ is —NH₃⁺Z⁻.

In some aspects, Z⁻ is

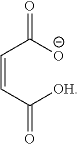

In some aspects, R³ is

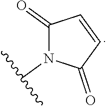 [SMILES: *N1C(=O)C=CC1=O]

Representative crosslinking agents of Formula I include, but are not limited to:

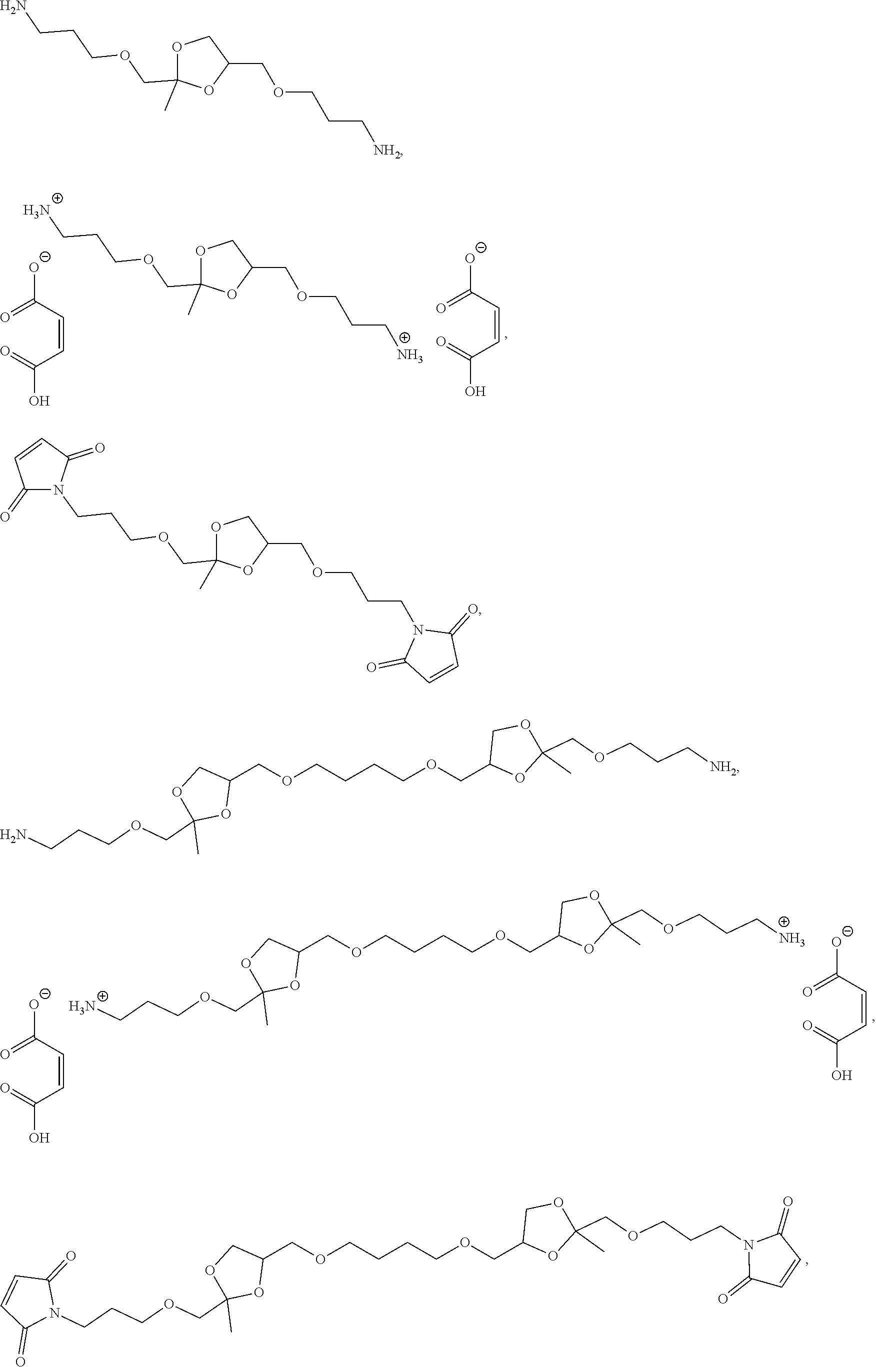

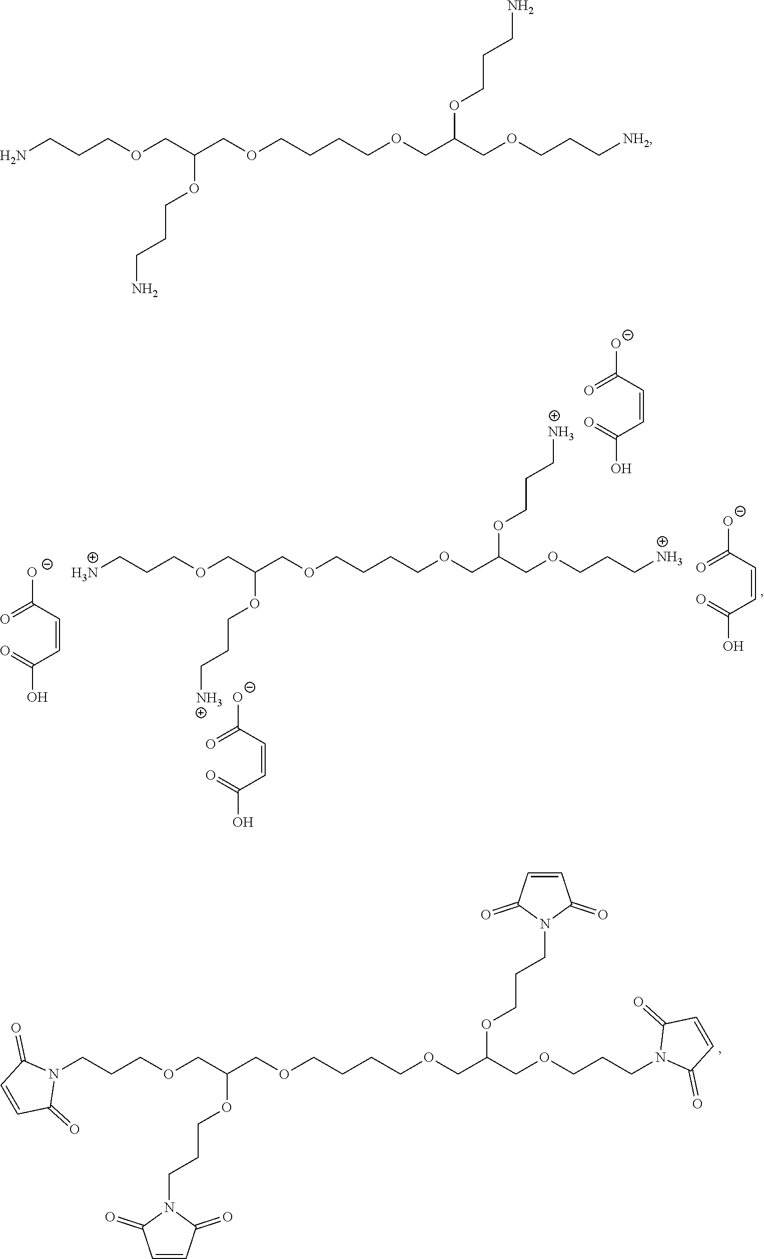

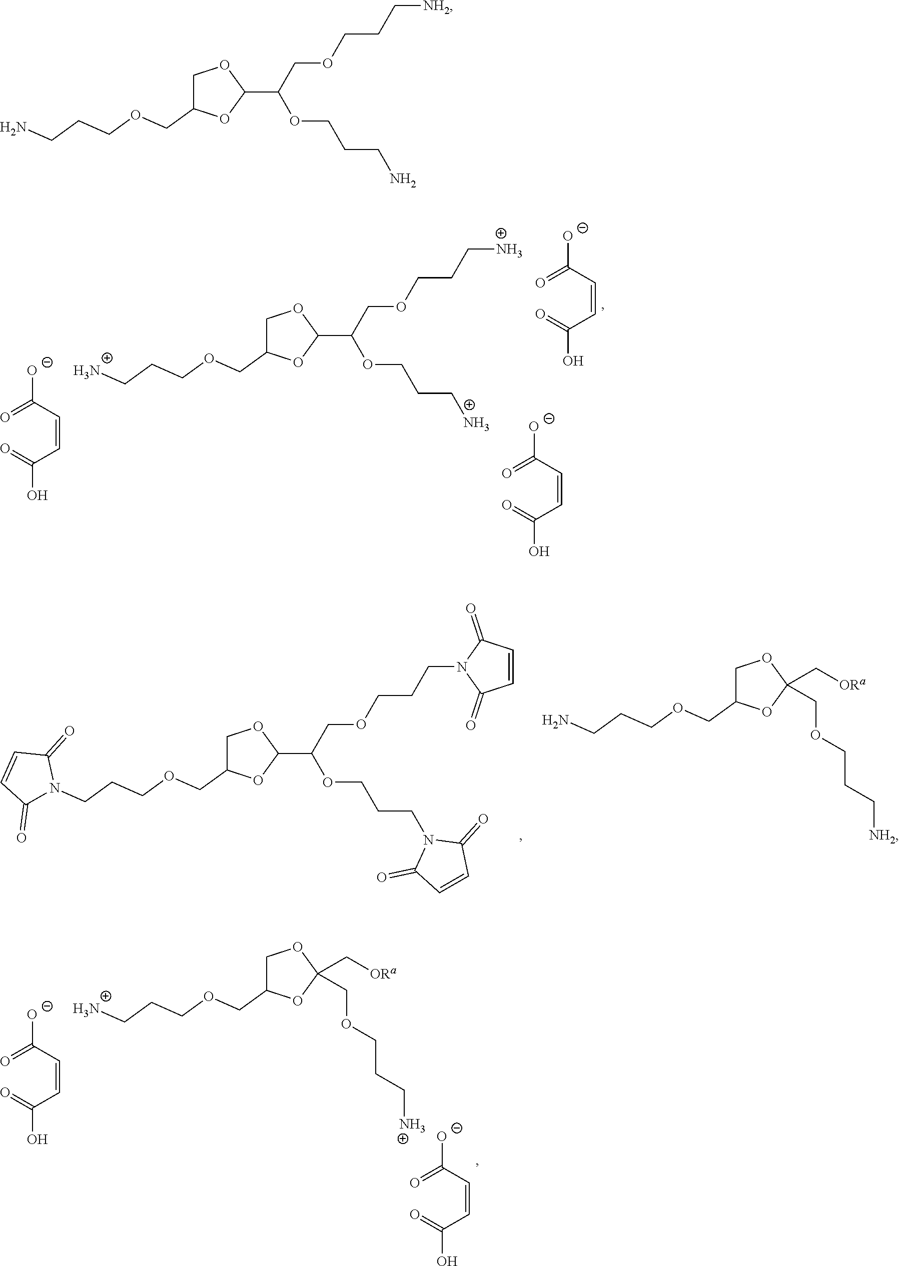

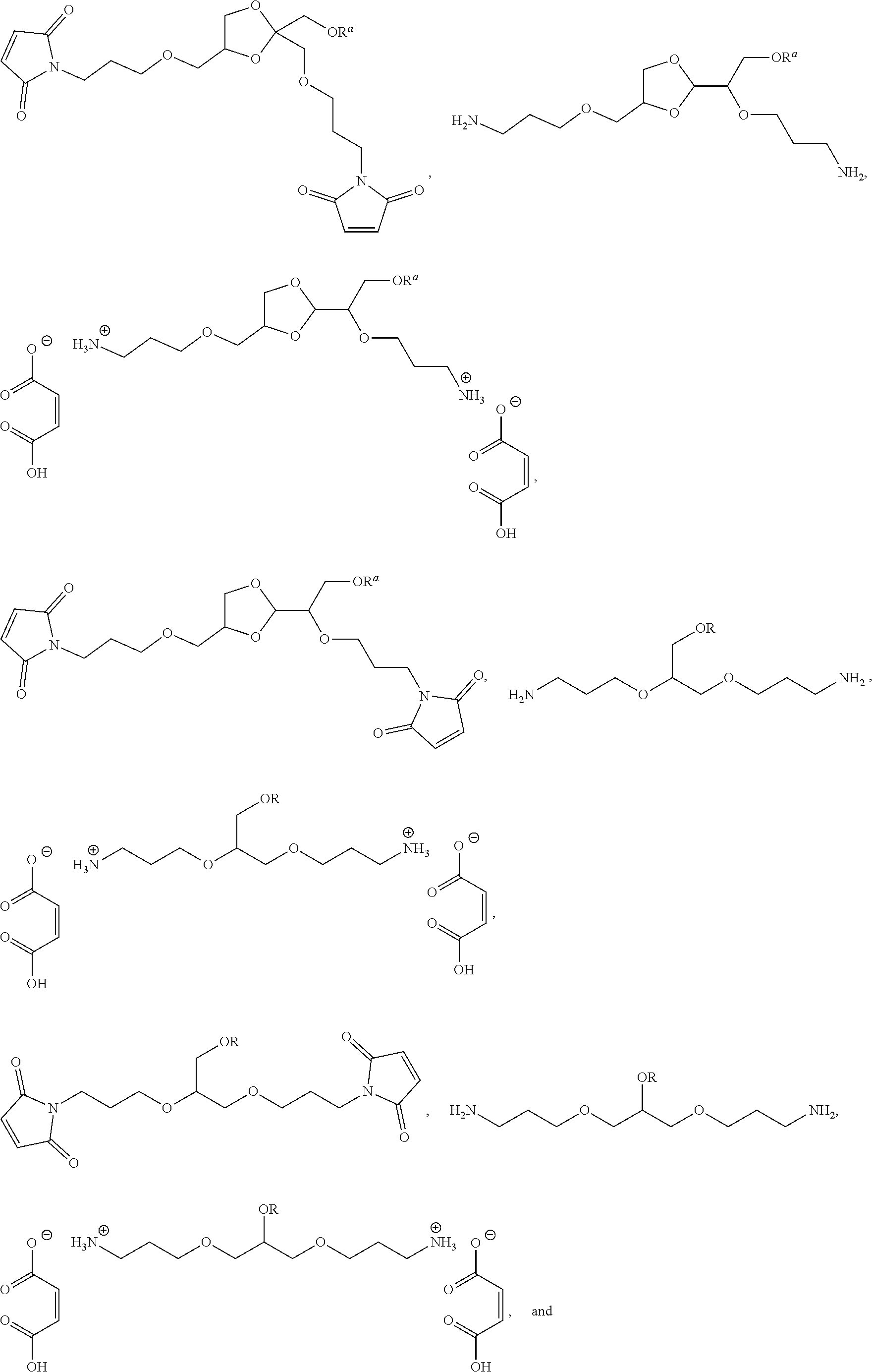

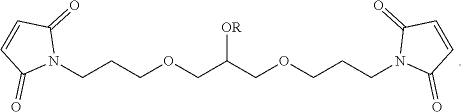

In another aspect, a crosslinking agent is provided of Formula II

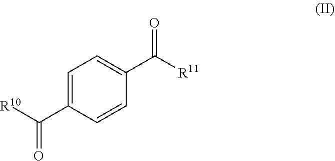

wherein:

R¹⁰ is selected from —OR¹ and —NR¹R¹;

R¹¹ is selected from —OR² and —NR²R²;

R¹ and R² are independently selected at each occurrence from

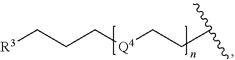

to wherein n is selected at each occurrence from 0, 1, 2, or 3;

R¹′ is independently selected from hydrogen or R¹;

R²′ is independently selected from hydrogen or R²;

R³ is selected at each occurrence from

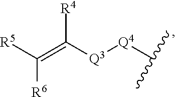

—NH₂, —NH₃⁺Z⁻, and

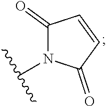 [SMILES: *N1C(=O)C=CC1=O]

Q³ is absent or independently selected at each occurrence from —C(═O)- and —S(═O)₂—;

Q⁴ is independently selected at each occurrence from —O— and —NH—;

R⁴, R⁵, and R⁶ are each independently selected at each occurrence from hydrogen, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;

Z⁻ is selected at each occurrence from a halide,

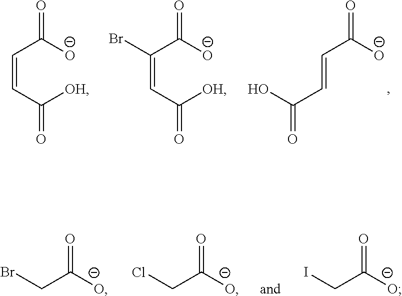

X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O-(C₀-C₃ alkyl)-, R^{x}S—(C₀-C₃ alkyl)-, (R^{x}R^{y}N)—(C₀-C₃ alkyl)-, R^{x}O—C(O)-(C₀-C₃ alkyl)-, R^{x}—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)-(R^{x}N)—(C₀-C₃ alkyl)-, R^{z}S(O)²-O—(C₀-C₃ alkyl)-, R_{z}S(O)₂—(R^{x}N)—(C₀-C₃ alkyl)-, R_{z}C(O)-(C₀-C₆ alkyl)-, R^{z}S(O)—(C₀-C₃ alkyl)-, and R^{z}S(O)²—(C₀-C₃ alkyl)-;

R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C³ alkyl)-, (5- to 10-membered monocyclic or bicyclic heteroaryl)-(C₀-C₃ alkyl)-, each of which may be optionally substituted with one or more Y groups as allowed by valency;

R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and

Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some aspects of Formula II, R¹⁰ is —OR¹. In some aspects of Formula II, R¹⁰ is —NR¹R¹′.

In some aspects of Formula II, R¹′ is hydrogen. In some aspects of Formula II, R¹′ is to R¹.

In some aspects of Formula II, R¹¹ is —OR². In some aspects of Formula II, R¹¹ is —NR²R²′.

In some aspects of Formula II, R²′ is hydrogen. In some aspects of Formula II, R²′ is R².

In some aspects of Formula II, R¹ and R² are each

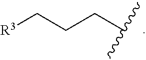

In some aspects of Formula II, R¹ and R² are each

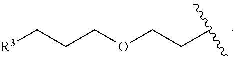

In some aspects of Formula II, R¹ and R² are each

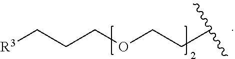

In some aspects of Formula II, R¹ and R² are each

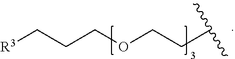

In some aspects of Formula II, R³ is

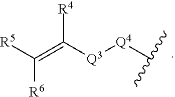

In some aspects of Formula II, Q³ is absent. In some aspects of Formula II, Q³ is C(═O)—.

In some aspects of Formula II, Q⁴ is —O—. In some aspects of Formula II, Q⁴ is —NH—.

In some aspects of Formula II, R⁴, R⁵, and R⁶ are each hydrogen. In some aspects of Formula II, R⁵ is phenyl and R⁴ and R⁶ are each hydrogen. In some aspects of Formula II, R⁴ is methyl and R⁵ and R⁶ are each hydrogen.

In some aspects of Formula II, R³ is —NH₂. In some aspects of Formula II, R³ is —NH₃⁺Z⁻.

In some aspects of Formula II, Z⁻ is

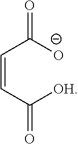

In some aspects of Formula II, R³ is

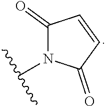 [SMILES: *N1C(=O)C=CC1=O]

Representative crosslinking agents of Formula II include, but are not limited to:

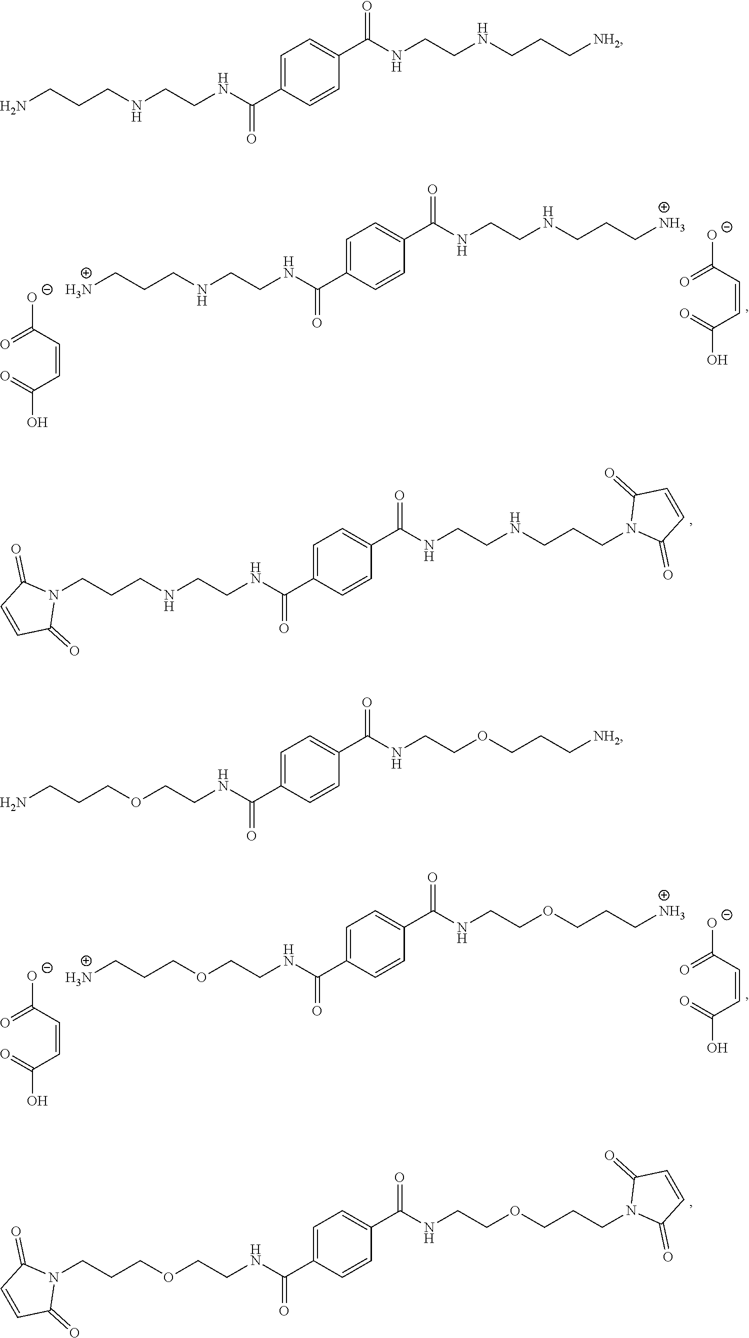

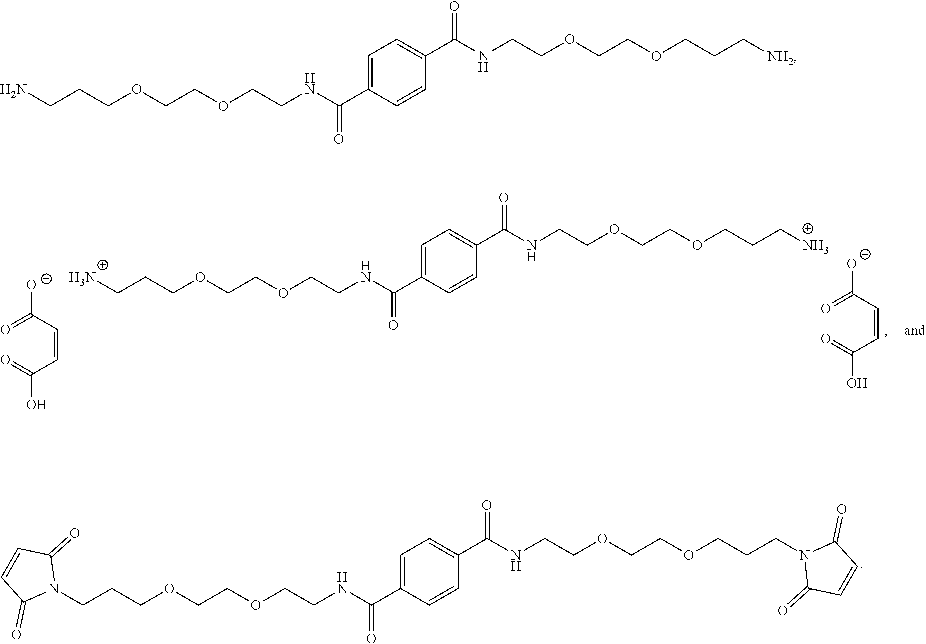 [SMILES: NCCCOCCOCCNC(=O)c1ccc(C(=O)NCCOCCOCCCN)cc1 ; O=C([O-])/C=C\C(=O)O ; [NH3+]CCCOCCOCCNC(=O)c1ccc(C(=O)NCCOCCOCCC[NH3+])cc1 ; O=C([O-])/C=C\C(=O)O ; O=C(NCCOCCOCCCN1C(=O)C=CC1=O)c1ccc(C(=O)NCCOCCOCCCN2C(=O)C=CC2=O)cc1]

In another aspect, a polymer composition is provided comprising at least one polymer crosslinking with a crosslinking agent described herein.

In another aspect, a resin composition is provided comprising at least one monomer and a crosslinking agent described herein.

The compounds described herein may find use in cosmetic and personal care formulations. Such formulations, as further described herein, are concerned with treating hair or skin. In some aspects, the formulations can rebuild latent disulfide bonds in hair or skin or may also react with free amines in the hair or skin to provide a conditioning effect.

The formulations contain one or more of the compounds described herein. The compound can be combined with one or more pharmaceutically acceptable carriers and/or excipients that are considered safe and effective for human hair, skin, and/or human scalp and may be administered to an individual's hair without causing undesirable side effects, such as burning, itching, and/or redness, or similar adverse reactions. The formulations may further contain an excipient that renders the formulations neutral pH or a pH ranging from about pH 3 to about pH 12, preferably from pH 5 to pH 8.

The compound is typically present in an amount ranging from about 0.01 wt % to about 50 wt % of the formulation, preferably from about 1 wt % to about 25 wt % of the formulation, more preferably from about 1 wt % to about 15 wt %, most preferably from about 1 wt % to about 10 wt %. When used in cosmetic and personal care formulations as described herein, the compound is typically stable in aqueous solution for a period of at least 2, 3, 4, 5, 6, 7, 8, 9, 10, 11, or 12 months or longer at a pH of 6 to 8 and a temperature of about 25-30° C. The excipient is typically present in an amount ranging from about 10 wt % to about 99.99 wt % of the formulation, preferably about 40 wt % to about 99 wt %, more preferably from about 80 wt % to about to about 99 wt %.

The formulations typically contain one or more cosmetically acceptable excipients. Cosmetically acceptable excipients include, but are not limited to, water, preservatives, antioxidants, chelating agents, sunscreen agents, vitamins, dyes, hair coloring agents, proteins, amino acids, natural extracts such as plant extracts, humectants, fragrances, perfumes, oils, emollients, lubricants, butters, penetrants, thickeners, viscosity modifiers, polymers, resins, hair fixatives, film formers, surfactants, detergents, emulsifiers, opacifying agents, volatiles, propellants, liquid vehicles, carriers, salts, pH adjusting agents (e.g., citric acid), neutralizing agents, buffers, hair conditioning agents, anti-static agents, anti-frizz agents, anti-dandruff agents, absorbents, and combinations thereof.

The formulations can contain at least two or more cosmetically acceptable excipients. In some forms, the formulations contain one or more compounds described herein, water, and optionally a preservative and/or fragrance.

The formulation for treating hair may be in any suitable physical form. Suitable forms include but are not limited to low to moderate viscosity liquids, lotions, milks, mousses, sprays, gels, creams, shampoos, conditioners, and the like. Suitable excipients, such as those listed above, are included or excluded from the hair care formulation depending on the form of use of the formulation (e.g., hair spray, cream, conditioner, or shampoo).

The formulation for treating skin may be in any suitable physical form. Suitable forms include but are not limited to low to moderate viscosity liquids, lotions, milks, mousses, sprays, gels, creams, ointments, and the like. Suitable excipients, such as those listed above, are included or excluded from the skin formulation depending on the form of use of the formulation (e.g., lotion, gel, ointment, or cream).

Surfactants are surface-active agents that are able to reduce the surface tension of water and cause the formulation to slip across or onto the skin or hair. Surfactants also include detergents and soap. The surfactants may be amphoteric, anionic, or cationic. Suitable surfactants that may be used in the formulation include, but are not limited to, 3-aminopropane sulfonic acid, almond amide, almond amidopropyl betaine, almond amidopropylamine oxide, aluminum hydrogenated tallow glutamate, aluminum lanolate, aminoethyl sulfate, aminopropyl lauryl glutamine, ammonium C₁₂₋₁₅ alkyl sulfate, ammonium C₁₂₋₁₅ pareth sulfate, ammonium C₁₂₋₁₆ alkyl sulfate, ammonium C₉₋₁₀ perfluoroalkylsulfonate, ammonium capryleth sulfate, ammonium capryleth-3 sulfate, ammonium monoglyceride sulfate, ammonium sulfate, ammonium isothionate, ammonium cocoyl sarcosinate, ammonium cumene sulfonate, ammonium dimethicone copolyol sulfate, ammonium dodecylbenzenesulfonate, ammonium isostearate, ammonium laureth sulfate, ammonium laureth-12 sulfate, ammonium laureth-5 sulfate, ammonium laureth-6 carboxylate, ammonium laureth-7 sulfate, ammonium laureth-8 carboxylate, ammonium laureth-9 sulfate, ammonium lauroyl sarcosinate, ammonium lauryl sulfate, ammonium lauryl sulfosuccinate, ammonium myreth sulfate, ammonium myristyl sulfate, ammonium nonoxynol-30 sulfate, ammonium nonoxynol-4 sulfate, ammonium oleate, ammonium palm kernel sulfate, ammonium polyacrylate, ammonium stearate, ammonium tallate, ammonium xylene sulfonate, ammonium xylene sulfonate, amp-isostearoyl gelatin/keratin amino acids/lysine hydroxypropyltrimonium chloride, amp-isostearoyl hydrolyzed collagen, apricot kernel oil PEG-6 esters, apricot amide, apricot amidopropyl betaine, arachideth-20, avocadamide, avocadamidopropyl betaine, babassuamide, babassuamidopropyl betaine, babassuamidopropylamine oxide, behenalkonium chloride, behenamide, behenamide, behenamidopropyl betaine, behenamine oxide, sodium laureth sulfate, sodium lauryl sulfate, a polyoxyether of lauryl alcohol or ceteareth-20, or combinations thereof. Suitable anionic surfactants include, but are not limited to, those containing carboxylate, sulfonate, and sulfate ions. Examples of anionic surfactants include sodium, potassium, ammonium of long chain alkyl sulfonates, and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol mono stearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine. More than one surfactant may be included in the formulation. The surfactants are optionally included in an amount ranging from about 0.1% to about 15% by weight of the formulation, preferably about 1% to about 10% by weight of the formulation.

Emollient refers to a material that protects against wetness or irritation, softens, soothes, coats, lubricates, moisturizes, protects, and/or cleanses the skin. Suitable emollients for use in the formulations include but are not limited to a silicone compound (e.g., dimethicone, cyclomethicone, dimethicone copolyol, or a mixture of cyclopentasiloxane and dimethicone/vinyldimethicone cross polymer, cyclopentasiloxane polysilicone), polyols such as sorbitol, glycerin, propylene glycol, ethylene glycol, polyethylene glycol, caprylyl glycol, polypropylene glycol, 1,3-butane diol, hexylene glycol, isoprene glycol, xylitol; ethylhexyl palmitate; a triglyceride such as caprylic/capric triglyceride and fatty acid ester such as cetearyl isononanoate or cetyl palmitate. In a specific aspect, the emollient is dimethicone, amidodimethicone, dimethiconol, cyclopentasiloxane, potassium dimethicone PEG-7 panthenyl phosphate, or a combination thereof. More than one emollient may be included in the formulation. The emollient is optionally included in an amount ranging from about 0.5% to about 15% by weight of the formulation, preferably from about 1% to about 10% by weight of the formulation.

The formulations may also contain one or more emulsifiers. Suitable emulsifiers include, but are not limited to, copolymers of an unsaturated ester and styrene sulfonate monomer, cetearyl alcohol, glyceryl ester, polyoxyethylene glycol ether of cetearyl alcohol, stearic acid, polysorbate-20, ceteareth-20, lecithin, glycol stearate, polysorbate-60, or polysorbate-80, or combinations thereof. More than one emulsifier may be included in the formulation. The emulsifier is optionally included in an amount ranging from about 0.05% to about 15% by weight of the formulation, preferably from about 0.1% to about 10% by weight of the formulation.

One or more preservatives may be included in the formulations to prevent microbial growth in the formulations. Suitable preservatives include, but are not limited to, glycerin-containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), potassium sorbate, and/or grapefruit seed extract, or combinations thereof. More than one preservative may be included in the formulation. Other preservatives are known in the cosmetics industries and include salicylic acid, DMDM Hydantoin, Formaldehyde, Chlorphenism, Triclosan, Imidazolidinyl Urea, Diazolidinyl Urea, Sorbic Acid, Methylisothiazolinone, Sodium Dehydroacetate, Dehydroacetic Acid, Quatemium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol). The preservative is optionally included in an amount ranging from about 0.1% to about 5% by weight of the formulation, preferably from about 0.3% to about 3% by weight of the formulation. Preferably, the formulations are paraben free.

One or more conditioning agents may be included in the formulations. Suitable conditioning agents include, but are not limited to, silicone-based agents (e.g., silicone quaternium-8), panthenol, hydrolyzed wheat and/or soy protein, amino acids (e.g., wheat amino acids), rice bran wax, meadowfoam seed oil, mango seed oil, grape seed oil, jojoba seed oil, sweet almond oil, hydroxyethyl behenamidopropyl dimonium chloride, aloe leaf extract, aloe barbadensis leaf juice, phytantriol, panthenol, retinyl palmitate, behentrimonium methosulfate, cyclopentasiloxane, quatemium-91, stearamidopropyl dimethylamine, and combinations thereof. The conditioning agent(s) is optionally included in an amount ranging from about 0.1% to about 5% by weight of the formulation, preferably from about 0.3% to about 3% by weight of the formulation.

Diluent, as used herein, refers to a substance(s) that dilutes a compound described herein. Water is the preferred diluent. The formulations typically contain greater than one percent (wt) water, preferably greater than five percent (wt) water, more preferably greater than 50% (wt) water, and most preferably greater than 80% (wt) water. Alcohols, such as ethyl alcohol and isopropyl alcohol, may be used at low concentrations (about 0.5% by weight of the formulation) to enhance hair or skin penetration and/or reduce odor.

The formulations may contain one or more viscosity modifying agents, such as viscosity increasing agents. Classes of such agents include, but are not limited to, viscous liquids, such as polyethylene glycol, semisynthetic polymers, such as semisynthetic cellulose derivatives, synthetic polymers, such as carbomers, poloxamers, and polyethyleneimines (e.g., PEI-10), naturally occurring polymers, such as acacia, tragacanth, alginates (e.g., sodium alginate), carrageenan, vegetable gums, such as xanthan gum, petroleum jelly, waxes, particulate associate colloids, such as bentonite, colloidal silicon dioxide, and microcrystalline cellulose, surfactants, such as PPG-2 hydroxyethyl coco/isostearamide, emulsifiers, such as disteareth-75 IPDI, and salts, such as sodium chloride, and combinations thereof.

The formulations may contain one or more antioxidants. Examples include, but are not limited to, tocopheryls, BHT, ascorbic acid, Camellia sinensis leaf extract, ascorbyl palmitate, magnesium ascorbyl phosphate, carotenoids, resveratrol, triethyl citrate, arbutin, kojic acid, tetrahexydecyl ascorbate, superoxide dismutase, zinc, sodium metabisulfite, lycopene, ubiquinone, and combinations thereof.

The formulations may contain one or more opacifying agents. Opacifying agents are added to the formulations to make them opaque. Suitable opacifying agents include, but are not limited to, glycol distearate and ethoxylated fatty alcohols.

The formulation may be in the form of a spray. The spray typically includes one or more compounds described herein and a cosmetically acceptable carrier. In some aspects, the carrier is water or a water and alcohol mixture. The spray formulation optionally includes an antioxidant, sunscreen agent, vitamin, protein, peptide, plant extract, humectant, oil, emollient, lubricant, thickener, hair conditioning agent, polymer, and/or surfactant. Preferably, the spray formulation includes a preservative. In some aspects, the formulation includes a fragrance. In some aspects, the formulation includes a surfactant. In some aspects, the formulation contains water, fragrance, a preservative, and a compound described herein. In some aspects, the formulation contains water, fragrance, a preservative, and a compound described herein. In some aspects, the formulation contains water, a preservative, fragrance, a compound described herein, and an anti-static agent. In some aspects, the formulation contains water, a preservative, fragrance, a compound described herein, and a hair conditioning agent. In some aspects, the formulation contains water, a preservative, fragrance, a compound described herein, and a surfactant.

The hair spray formulations may be dispensed from containers that include aerosol dispensers or pump spray dispensers. Such dispensers are known in the art and are commercially available from a variety of manufacturers. When the hair spray formulation is dispensed from a pressurized aerosol container, a propellant may be used to force the composition out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed. The amount of propellant may range from about 10% to about 60% by weight of the formulation. The propellant may be separated from the hair repair formulation as in a two-compartment container. Other suitable aerosol dispensers are those characterized by the propellant being compressed air, which can be filled into the dispenser using a pump or equivalent device prior to use. Conventional non-aerosol pump spray dispensers, i.e., atomizers, may also be used to apply the hair-strengthening formulation to the hair.

The formulation may be in the form of a conditioner. The conditioner typically includes a compound described herein in a suitable carrier. Additionally, the conditioner may include cationic polymers derived from polysaccharides, for example, cationic cellulose derivatives, cationic starch derivatives, cationic guar derivatives and cationic locust bean gum derivatives, synthetic cationic polymers, mixtures or combinations of these agents. The formulation may comprise other synthetic or natural polymers or polymers derived from biological preparation processes, which are functionalized, where appropriate, for example, with cationic or neutral groups. These polymers may have a stabilizing or strengthening action on the compositions and/or a conditioning action (deposition on the surface of the skin or the hair). The compound may be included in any suitable concentration. Typical concentrations of the compound in the conditioner range from small amounts, such as approximately 0.01% (wt), preferably at least 0.1% (wt), to large amounts, such as up to 50% (wt). Preferably the conditioner contains the compound in a concentration ranging from 0.1% (wt) to 5% (wt), more preferably from 0.1% wt to 3% (wt). While greater concentrations of a compound described herein could be present in the conditioner, they are generally not needed to achieve the desired results.

The hair repair formulation may be in the form of a shampoo. The shampoo typically includes the compound described herein in a suitable carrier. The compound may be included in any suitable concentration. Typical concentrations of the compound in the shampoo range from small amounts, such as approximately 0.01% (wt), preferably at least 0.1% (wt), to large amounts, such as up to 50% (wt). Preferably the shampoo contains the compound in a concentration ranging from 0.1% (wt) to 5% (wt), more preferably from 0.1% wt to 3% (wt). While greater concentrations of a compound described herein could be present in the shampoo, they are generally not needed to achieve the desired results. Additionally, the shampoo may include about 0.5% to about 20% of a surfactant material. Surfactants utilized in shampoo compositions are well-known in the art and are disclosed, for example, in U.S. Pat. Nos. 6,706,258 and 7,598,213.

The formulation may be in the form of a cream. The cream typically includes the compound described herein in a suitable carrier. The compound may be included in any suitable concentration. Typical concentrations of the compound in the cream range from small amounts, such as approximately 0.01% (wt), preferably at least 0.1% (wt), to large amounts, such as up to 50% (wt). Preferably the cream contains the compound in a concentration ranging from 0.1% (wt) to 5% (wt), more preferably from 0.1% wt to 3% (wt). While greater concentrations of a compound described herein could be present in the cream, they are generally not needed to achieve the desired results. Additionally, the cream may include an oil, a hair conditioning agent, and/or a thickening agent. The cream may also include a fragrance, a plant extract, and/or a surfactant. The cream may be packaged in a tube, tub, bottle, or other suitable containers.

In some aspects, a liquid binding formulation is provided, which is mixed at the time of use with a second formulation, such as a coloring or highlighting formulation. In these aspects, the liquid binding formulation may contain any suitable concentration of a compound described herein in a suitable carrier, typically a diluent, such as described above. The concentration of the compound is suitable to provide a mixture with the appropriate final volume and concentration of the compound. For example, a liquid binding formulation can contain a concentration of the compound ranging from about 5% (wt) to about 50% (wt) or greater. In a preferred aspect, the liquid binding formulation contains about 20% (wt) of the compound. The terms “highlighting” and “bleaching” are used synonymously herein. For highlighting applications, prior to use, a sufficient volume of a liquid binding formulation is mixed with a sufficient volume of a highlighting formulation to form a highlighting mixture having the desired concentration of the compound. Typical concentrations of the compound in the highlighting mixture range from small amounts, such as approximately at least 0.01% (wt), preferably at least 0.1% (wt), to large amounts, such as up to 50% (wt). Preferably the highlighting mixture contains the compound in a concentration ranging from 0.1% (wt) to 5% (wt), more preferably from 0.1% wt to 3% (wt). While greater concentrations of a compound described herein could be present in the highlighting mixture, they are generally not needed to achieve the desired results. Alternatively, two separate formulations are applied, such as a first formulation containing bleach (i.e., the highlighting formulation) and a second formulation containing a compound described herein (i.e., the binding formulation) in an effective amount to covalently bind the free thiol groups. The highlighting formulation may be applied first, which produces free thiol groups in hair. Subsequently, the second binding formulation may be applied to bind the free thiol groups.

Methods of using the cosmetic and personal care formulations described herein are also provided. In one aspect, prior to treatment with a compound described herein, the hair has been damaged, and the thiol groups in the hair are free thiols. The compound can be applied to the hair to bind the free thiol groups. Preferably, the compound is applied at least within one week of the hair being damaged, preferably within three days, more preferably within two days, and most preferably, the same day. Optionally, the hair may be shampooed and/or conditioned prior to applying the binding formulation. Alternately, the hair may only be rinsed with water prior to application of the binding formulation. Subsequent to shampooing, conditioning, and/or rinsing the hair, the binding formulation is applied to the hair. Alternately, the hair does not have to be washed or rinsed prior to application of the binding formulation. In this aspect, the binding formulation is applied to dry hair. The binding formulations may be used as a daily conditioning treatment for hair. Typically, the amount of binding formulation applied is sufficient to saturate the hair. The binding formulation may be applied to the hair as a single application, or application of a compound described herein may be repeated one or more times. Typically, the amount of binding formulation applied in each application is sufficient to saturate the hair. The volume of binding formulation applied to the hair in each application may be about 1 to about 100 mL per person, depending on the length and volume of hair. In some aspects, application of the compound could be repeated immediately (e.g., within about 10 to 15 seconds) or between about one and five minutes, greater than five minutes, between about five and ten minutes, greater than ten minutes, between about ten and twenty (20) minutes after the first application. Preferably, the hair is washed or rinsed subsequent to the application of the binding formulation. The hair may be rinsed and subsequently washed immediately (e.g., within 10, 15, 25, 30, 45, 60 seconds (one minute), two minutes, three minutes, four, or five minutes following application) after final application of a compound described herein. Alternatively, the hair may be rinsed and washed within about 30 minutes following application, preferably between about 5 minutes and about 20 minutes, more preferably about 10 minutes after the final application of the compound to the hair, depending on the hair type. Alternately, the hair does not have to be washed or rinsed subsequent to application of the binding formulation. The compound covalently binds latent free thiols in the hair. The thiols remain bound for at least one week, preferably for at least one month following application of the compound. The thiols may remain bound for longer periods of time, such as for about two months or more following application of the compound. The binding reaction is a stable reaction, such that the thiols may remain bound even if subjected to a hair coloring treatment (simultaneous or subsequent to the binding reaction).

In one aspect, prior to treatment with a compound described herein, the hair has been subjected to a reducing agent used for waving (also referred to herein as hair perming or permanent waves), curling, and/or straightening of the hair. The first step in waving, curling, or straightening hair is breaking the cysteine disulfide bonds to form free thiol moieties. The process for breaking the cysteine disulfide bonds is via application of a reducing agent. The process for applying the reducing agent involves following normal perming or hair straightening procedures that are known to those skilled in the art. For example, to perm a hair, the hair is first washed and set on perm rods of various sizes. Second, a reducing agent, such as thioglycolate reducing solution or lotion, is applied to the hair. The hair is allowed to set for a specified period of time, and then the thioglycolate solution is rinsed from the hair. The application of hydrogen peroxide in this process is optional. In some processes, such as when treating previously chemically treated hair, hydrogen peroxide is generally not used. In other processes, such as when perming virgin hair, hydrogen peroxide may be added. In these aspects, hydrogen peroxide is typically added after the reducing agent is rinsed out. Then the hydrogen peroxide is rinsed from the hair prior to adding the compound. Subsequent to the reducing treatment, one or more of the compound or a formulation thereof is applied to the hair. Although the compound is typically applied on the same day as treatment with the reducing agent, it may be applied later, such as within 1 to 2 weeks following treatment with the reducing agent. Typically, the amount of binding formulation applied is sufficient to saturate the hair. The compound is generally rinsed and shampooed from the hair after the desired level of hair waving, curling, or straightening is achieved. In some aspects, the compound is rinsed from the hair immediately (e.g., within 10, 15, 25, 30, 45, or 60 seconds following application) following the final application of the compound. Alternatively, the hair may be rinsed and washed within about 30 minutes following application, preferably between about 5 minutes and about 20 minutes, more preferably about 10 minutes after the final application of the compound to the hair, depending on the hair type. The compound can be rinsed from the hair within 10, 15, 25, 30, 45, or 60 seconds from the hair after application and still achieve the desired level of hair waving, curling, or straightening. The compound described herein may be applied to the hair as a single application, or application of the compound may be repeated one or more times. Typically, the amount of binding formulation applied in each application is sufficient to saturate the hair. In some aspects, the volume of binding formulation applied to the hair in each application is about 1 to about 10 mL per perm rod. In some aspects, application of the compound could be repeated immediately (e.g., within to 10 to 15 seconds) or approximately 1, 5, 7.5, 10, 12.5, 15, 17.5, or 20 minutes after the first application. In some aspects, the second application is about 7 minutes to about 10 minutes after the first application. The compound is rinsed from the hair after its application. The hair may be rinsed and washed immediately (e.g., within 10 to 15 seconds following application) after final application of the compound. Alternatively, the hair may be rinsed and washed about 10 minutes or later after the final application of the compound, such as about 15 minutes to about 30 minutes, preferably about 20 minutes after repeated application of the compound to the hair. The compounds described herein covalently bind the free thiols in the hair. The thiols remain bound for at least one week, two weeks, three weeks, four weeks, one month, two months, or more. The compounds described herein are generally washed from the individual's hair on the same day as they are applied. In contrast, traditional perms which use only hydrogen peroxide (and do not involve the addition of a crosslinking agent) are generally not washed for at least 48 hours following application (washing the hair prior to 48 hours following a traditional permanent treatment may result in a significant loss in the amount of curl in the hair and/or cause damage to the hair). The compositions described herein improve hair quality, such as appearance (e.g., sheen) and feel, increase dry strength (e.g., tensile strength), and decrease hair breakage when the hair is subjected to subsequent treatments, such as coloring. In some aspects, hair breakage decreases by 5, 10, 15, 20, 25, 30, 35, 40, 45, 50, 55, 60, 65, 70, or 75% or higher after treatment with the compounds described herein compared to untreated hair from the same individual. Hair breakage is a significant problem encountered during coloring and other treatments.

The coloring formulation is generally applied to an individual's hair following normal hair coloring procedures that are known to those skilled in the art. Typically, hair color treatments include two complementary processes: bleaching the hair's natural pigment and/or other artificial pigments present in the hair, and diffusion of dye precursors into the hair, followed by coupling reactions that result in the formation of chromophores within the hair shaft, which are too large to diffuse out of the hair. The hair coloring formulation may be a highlighting formulation, such as formed by mixing bleach powder and developer. More complex colors may contain several precursors and many couplers and may involve multiple reactions. The dye precursors may contain several ingredients, each with different functions. The first ingredient is usually an alkalizing agent (usually ammonia and/or an ammonia substitute, such as monoethanolamine [MEA]). The alkalizing agent serves a number of roles in the hair colorant process, including swelling the hair fiber to aid in diffusion of the dye precursors. The dye precursors generally include p-diamines and p-aminophenols. Precursors are oxidized to active intermediates once they have penetrated the hair shaft. Intermediates then react with color couplers to create wash-resistant dyes. More specifically, the intermediates, in the presence of an oxidant, couple with another oxidation dye intermediate molecule to form a large fused ring color compound within the hair shaft. since the fused ring product is too large to penetrate the hair shaft. Couplers modify the color produced by the oxidation of precursor compounds. The primary difference between demi-permanent and permanent products is the alkalizing agent and the concentration of peroxide. The cuticle does not swell as greatly with demi-permanent dyes, making dye penetration less efficient compared to permanent coloring products. Several coloring formulations use a reducing agent, such as sodium bisulfate, to break disulfide bonds in the hair, allowing deeper penetration of the hair coloring dyes into the hair. Specifically, the method includes reducing some of the disulfide linkages of the cysteine in the hair shafts to thiol groups while breaking hydrogen bonds. The reducing process changes the chemical and cosmetic characteristics of the hair, which are undesirable. The hair dyeing process may be followed by a shampoo and conditioning treatment, a neutralizing rinse, or an acid-balanced shampoo containing, in addition to cationic or amphoteric surfactants, cation-active emollients, and quarternary polymers. Alternately, the hair dyeing process may be followed by application of the binding formulations described herein before a shampoo and/or conditioning treatment.

The binding formulation may be applied simultaneously with the hair coloring formulation or subsequently to the application of the hair coloring formulation. For example, the binding formulation may be mixed with the hair coloring treatment, and the mixture containing both the binding formulation and the hair coloring treatment may be applied to the hair. Alternatively, subsequent to coloring the hair, the binding formulation, or a formulation thereof, is applied to the hair. Although the compounds described herein are typically applied on the same day as the coloring treatment, they may be applied later, such as within 1 to 2 weeks following treatment with the reducing agent. Typically, the amount of binding formulation (or a mixture of the binding formulation and the hair coloring formulation) applied is enough to saturate the hair. The binding formulation may be applied to the hair as a single application, or application of the compound may be repeated one or more times. Typically, the amount of binding formulation applied in each application is sufficient to saturate the hair. The volume of binding formulation applied to the hair in each application may be about 1 to about 100 mL per person, depending on the length and volume of hair. In some aspects, application of the compound could be repeated immediately (e.g., within 10 to 15 seconds) or approximately 1, 5, 7.5, 10, 12.5, 15, 17.5, or 20 minutes after the first application.

The compound can be rinsed and shampooed from the hair immediately following application, for example, within 10, 15, 25, 30, 45, or 60 seconds, or two, three, four, or five minutes after application. Alternatively, the compound may be rinsed from the hair within about 30 minutes following application, preferably between about 5 minutes and about 20 minutes, more preferably about 10 minutes after application of the compound to the hair, depending on hair type. If the binding formulation is combined with the hair coloring treatment and applied as a mixture to the hair, then the mixture remains on the hair as long as needed for the hair coloring treatment. Typically the mixture is applied for approximately 10 minutes. The mixture is removed from the hair in accordance with standard methods for hair coloring treatments, e.g., rinse and shampoo, approximately 10 minutes after applying the mixture. The binding formulation is rinsed from the hair after its application. The hair may be rinsed and subsequently washed immediately (e.g., within 10 to 15 seconds following application) after final application of the compound. Preferably, the hair is rinsed and/or washed about 10 minutes or later after the final application of the compound, such as about 15 minutes to about 30 minutes, optionally about 20 minutes after repeated application of the compound to the hair. The compound covalently binds the free thiols in the hair. The thiols remain bound for at least one week, two weeks, three weeks, four weeks, one month, or two months, or more. The compounds are generally washed from the individual's hair on the same day as they are applied. In contrast, traditional perms which use only hydrogen peroxide (and do not involve the addition of a crosslinking agent) are generally not washed for at least 48 hours following application (washing the hair prior to 48 hours following a traditional permanent treatment may result in significant loss in the amount of curl in the hair and/or cause damage to the hair). The compositions described herein improve hair quality, such as appearance (e.g., sheen) and feel, increase dry strength (e.g., tensile strength), and decrease hair breakage when the hair is subjected to subsequent treatments, such as coloring. In some aspects, hair breakage decreases by 5, 10, 15, 20, 25, 30, 35, 40, 45, 50, 55, 60, 65, 70, 75, 80, 85, or 90% or higher after treatment with the compound compared to untreated hair from the same individual. Hair breakage is a significant problem encountered during coloring and other treatments.

In another aspect, the following particular aspects of the present disclosure are also provided:

Aspect 1. A crosslinking agent of Formula I

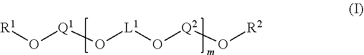

wherein:

m is 0 or 1;

Q¹ and Q², if present, are independently selected from:

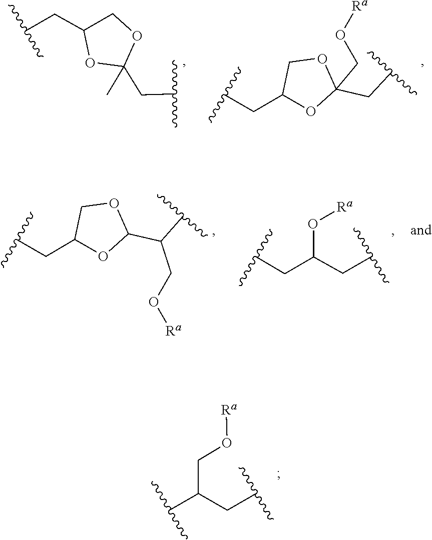

L¹, if present, is selected from C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;

R^{a} is independently selected at each occurrence from hydrogen, R¹, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;

R¹ and R² are independently selected at each occurrence from

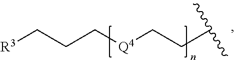

wherein n is selected at each occurrence from 0, 1, 2, or 3;

R³ is selected at each occurrence from

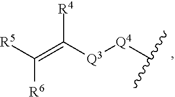

—NH₂, —NH₃⁺Z⁻, and

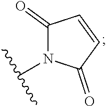 [SMILES: *N1C(=O)C=CC1=O]

Q³ is absent or independently selected at each occurrence from —C(═O)— and —S(═O)₂—;

Q⁴ is independently selected at each occurrence from —O— and —NH—;

R⁴, R⁵, and R⁶ are each independently selected at each occurrence from hydrogen, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;

Z⁻ is selected at each occurrence from a halide,

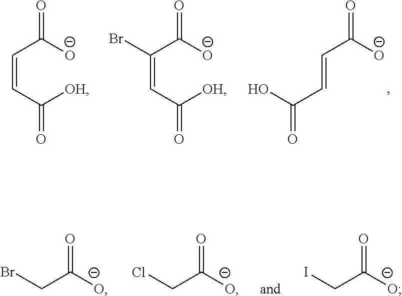

X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}—(C₀-C₃ alkyl)-, (R^{x}R^{y}N)—(C₀-C₃ alkyl)-, R^{x}O—C(O)—(C₀-C₃ alkyl)-, R^{x}—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;

R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and

Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

Aspect 2. The crosslinking agent of aspect 1, wherein m is 0.
Aspect 3. The crosslinking agent of aspect 1, wherein m is 1.
Aspect 4. The crosslinking agent of aspect 1 or aspect 3, wherein Q² is

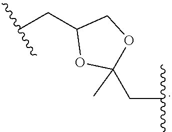 [SMILES: *CC1COC(C)(C*)O1]

Aspect 5. The crosslinking agent of aspect 1 or aspect 3, wherein Q² is

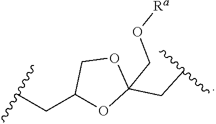

Aspect 6. The crosslinking agent of aspect 1 or aspect 3, wherein Q² is

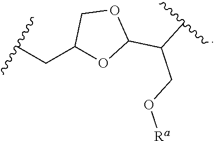

Aspect 7. The crosslinking agent of aspect 1 or aspect 3, wherein Q² is

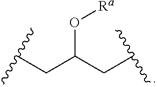

Aspect 8. The crosslinking agent of cl aspect aim 1 or aspect 3, wherein Q² is

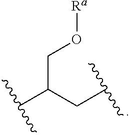

Aspect 9. The crosslinking agent of any one of aspects 1 and 3-8, wherein L¹ is C₁-C₁₀ alkyl.
Aspect 10. The crosslinking agent of any one of aspects 1 and 3-9, wherein L¹ is selected from C₁ alkyl, C₂ alkyl, C₃ alkyl, C₄ alkyl, Cs alkyl, C₆ alkyl, C₇ alkyl, C₈ alkyl, C₉ alkyl, and C₁₀ alkyl.
Aspect 11. The crosslinking agent of any one of aspects 1-10, wherein Q¹ is

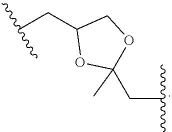 [SMILES: *CC1COC(C)(C*)O1]

Aspect 12. The crosslinking agent of any one of aspects 1-10, wherein Q¹ is

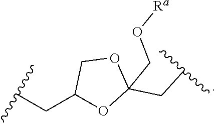

Aspect 13. The crosslinking agent of any one of aspects 1-10, wherein Q¹ is

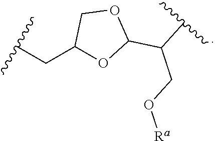

Aspect 14. The crosslinking agent of any one of aspects 1-10, wherein Q¹ is

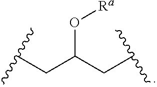

Aspect 15. The crosslinking agent of any one of aspects 1-10, wherein Q¹ is

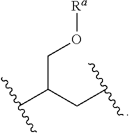

Aspect 16. The crosslinking agent of any one of aspects 1-15, wherein R^{a} is selected at each occurrence from hydrogen, C₁-C₁₀ alkyl, C₃-C₈ cycloalkyl, 3- to 8-membered monocyclic or bicyclic heterocycle, 6- to 10-membered monocyclic or bicyclic aryl, and 5-to 10-membered monocyclic or bicyclic heteroaryl.
Aspect 17. The crosslinking agent of any one of aspects 1-16, wherein R^{a} is R¹.
Aspect 16. The crosslinking agent of any one of aspects 1-15, wherein R¹ and R² are each

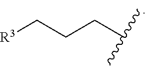

Aspect 17. The crosslinking agent of any one of aspects 1-15, wherein R¹ and R² are each

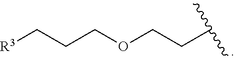

Aspect 18. The crosslinking agent of any one of aspects 1-15, wherein R¹ and R² are each

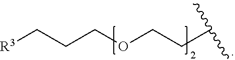

Aspect 19. The crosslinking agent of any one of aspects 1-15, wherein R¹ and R² are each

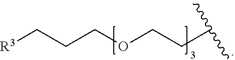

Aspect 20. The crosslinking agent of any one of aspects 1-19, wherein R³ is

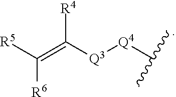

Aspect 21. The crosslinking agent of aspect 20, wherein Q³ is absent.
Aspect 22. The crosslinking agent of aspect 20, wherein Q³ is —C(═O)—.
Aspect 23. The crosslinking agent of any one of aspects 1-22, wherein Q⁴ is —O—.
Aspect 24. The crosslinking agent of any one of aspects 1-22, wherein Q⁴ is —NH—.
Aspect 25. The crosslinking agent of any one of aspects 20-24, wherein R⁴, R⁵, and R⁶ are each hydrogen.
Aspect 26. The crosslinking agent of any one of aspects 20-24, wherein R⁵ is phenyl, and R⁴ and R⁶ are each hydrogen.
Aspect 27. The crosslinking agent of any one of aspects 20-24, wherein R⁴ is methyl and R⁵ and R⁶ are each hydrogen.
Aspect 28. The crosslinking agent of any one of aspects 1-19, wherein R³ is —NH₂.
Aspect 29. The crosslinking agent of any one of aspects 1-19, wherein R³ is —NH₃⁺Z⁻.
Aspect 30. The crosslinking agent of aspect 29, wherein Z⁻ is

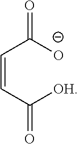

Aspect 31. The crosslinking agent of any one of aspects 1-19, wherein R³ is

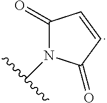 [SMILES: *N1C(=O)C=CC1=O]

Aspect 32. A crosslinking agent of Formula II

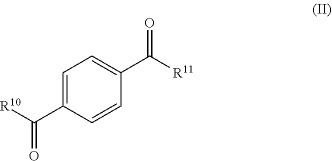

wherein:

R¹⁰ is selected from —OR¹ and —NR¹R¹′;

R¹¹ is selected from —OR² and —NR²R²′;

R¹ and R² are independently selected at each occurrence from

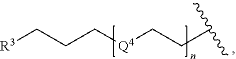

wherein n is selected at each occurrence from 0, 1, 2, or 3;

R¹′ is independently selected from hydrogen or R¹;

R²′ is independently selected from hydrogen or R²;

R³ is selected at each occurrence from

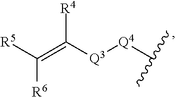

—NH₂, —NH₃⁺Z⁻, and

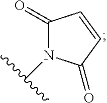 [SMILES: *N1C(=O)C=CC1=O]

Q³ is absent or independently selected at each occurrence from —C(═O)— and —S(═O)₂—;

Q⁴ is independently selected at each occurrence from —O— and —NH—;

R⁴, R⁵, and R⁶ are each independently selected at each occurrence from hydrogen, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;

Z⁻ is selected at each occurrence from a halide,

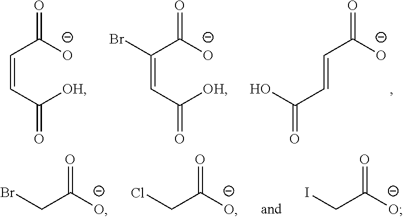

X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O-(C₀-C₃ alkyl)-, R^{x}—(C₀-C₃ alkyl)-, (R^{x}R^{y}N)-(C₀-C₃ alkyl)-, R^{x}O—C(O)—(C₀-C₃ alkyl)-, R^{x}—C(O)—(C₀-C₃ alkyl)-, (R^{x}R^{y}N) C(O)—(C₀-C₃ alkyl)-, to R^{x}O—S(O)2-(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂-(R^{x}N)—(C₀-C₃ alkyl)-, R^{z}C(O)—(C₀-C₆ alkyl)-, R^{z}S(O)—(C₀-C₃ alkyl)-, and R^{z}S(O)2—(C₀-C₃ alkyl)-; R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —ORₓ, —SR^{x}, and —NR^{x}R^{y}, each of which may be optionally substituted with one or more Y groups as allowed by valency; and

Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

Aspect 33. The crosslinking agent of aspect 32, wherein R¹⁰ is —OR¹.
Aspect 34. The crosslinking agent of aspect 32, wherein R¹⁰ is —NR¹R¹′.
Aspect 35. The crosslinking agent of aspect 34, wherein R¹′ is hydrogen.
Aspect 36. The crosslinking agent of aspect 34, wherein R¹′ is R¹.
Aspect 37. The crosslinking agent of any one of aspects 32-36, wherein R¹¹ is —OR².
Aspect 38. The crosslinking agent of any one of aspects 32-36, wherein R¹¹ is —NR²R²′.
Aspect 39. The crosslinking agent of aspect 38, wherein R²′ is hydrogen.
Aspect 40. The crosslinking agent of aspect 38, wherein R²′ is R².
Aspect 41. The crosslinking agent of any one of aspects 32-40, wherein R¹ and R² are each

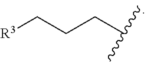

Aspect 42. The crosslinking agent of any one of aspects 32-40, wherein R¹ and R² are each

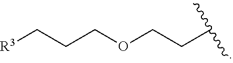

Aspect 43. The crosslinking agent of any one of aspects 32-40, wherein R¹ and R² are each

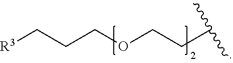

Aspect 44. The crosslinking agent of any one of aspects 32-40, wherein R¹ and R² are each

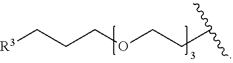

Aspect 45. The crosslinking agent of any one of aspects 32-40, wherein R³ is

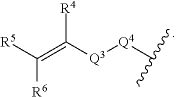

Aspect 46. The crosslinking agent of aspect 45, wherein Q³ is absent.
Aspect 47. The crosslinking agent of aspect 45, wherein Q³ is —C(═O)—.
Aspect 48. The crosslinking agent of any one of aspects 32-47, wherein Q⁴ is —O—.
Aspect 49. The crosslinking agent of any one of aspects 32-47, wherein Q⁴ is —NH—.
Aspect 50. The crosslinking agent of any one of aspects 45-49, wherein R⁴, R⁵, and R⁶ are each hydrogen.
Aspect 51. The crosslinking agent of any one of aspects 45-49, wherein R⁵ is phenyl and R⁴ and R⁶ are each hydrogen.
Aspect 52. The crosslinking agent of any one of aspects 45-49, wherein R⁴ is methyl and R⁵ and R⁶ are each hydrogen.
Aspect 53. The crosslinking agent of any one of aspects 32-52, wherein R³ is —NH₂.
Aspect 54. The crosslinking agent of any one of claims 32-52, wherein R³ is —NH₃⁺Z⁻.
Aspect 55. The crosslinking agent of aspect 54, wherein Z⁻ is

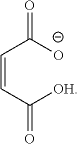

Aspect 56. The crosslinking agent of any one of aspects 32-52, wherein R³ is

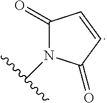 [SMILES: *N1C(=O)C=CC1=O]

Aspect 57. A polymer composition comprising at least one polymer crosslinked with a crosslinking agent of any one of aspects 1-56.
Aspect 58. An article formed from a polymer composition of aspect 57.
Aspect 59. A resin composition comprising at least one monomer and a crosslinking agent of any one of aspect 1-56.
Aspect 60. A personal care composition comprising a crosslinking agent of any one of aspects 1-56.
Aspect 61. The personal care composition of aspect 60, wherein the composition is a haircare product.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### EXAMPLES

The following examples are put forth so as to provide those of ordinary skill in the aclt with a complete disclosure and description of how the compounds, compositions, articles, devices, or methods claimed herein are made and evaluated and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

Representative compounds of the present disclosure are prepared according to the synthetic processes provided in the below schemes:

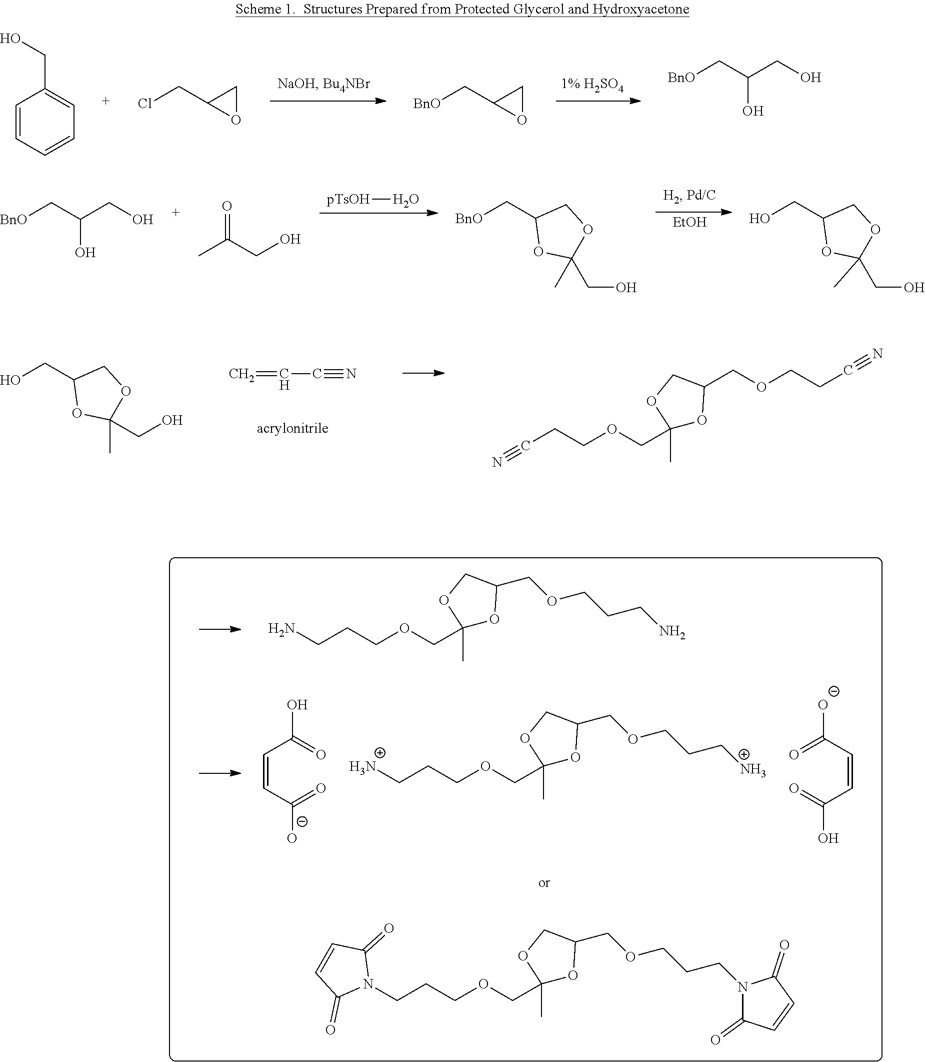

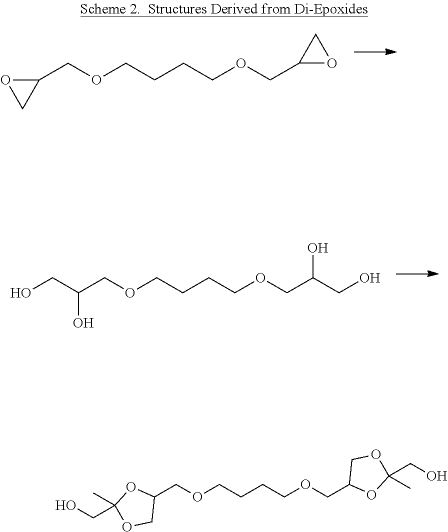 [SMILES: C(CCOCC1CO1)COCC1CO1 ; OCC(O)COCCCCOCC(O)CO ; CC1(CO)OCC(COCCCCOCC2COC(C)(CO)O2)O1]

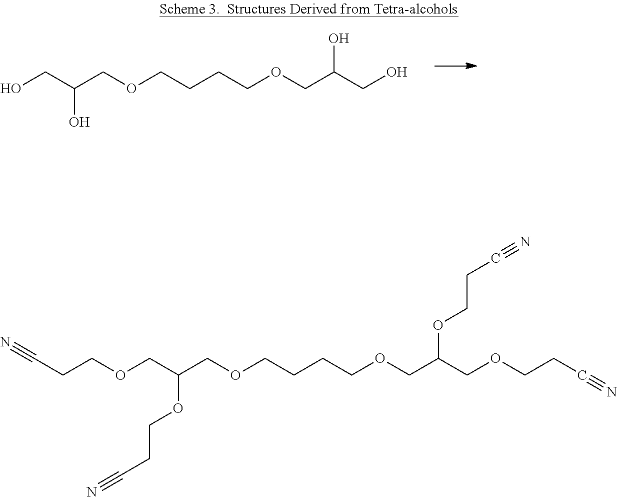 [SMILES: OCC(O)COCCCCOCC(O)CO ; N#CCCOCC(COCCCCOCC(COCCC#N)OCCC#N)OCCC#N]

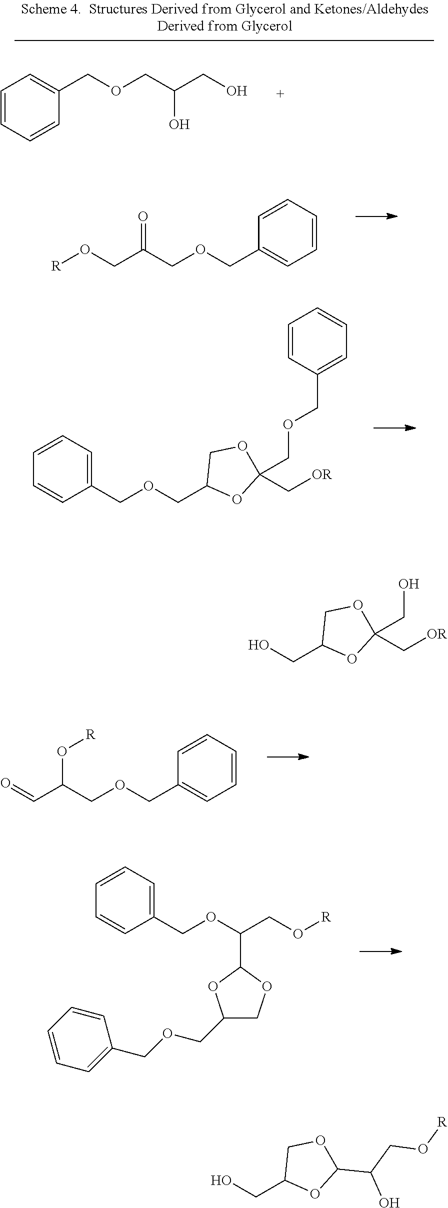

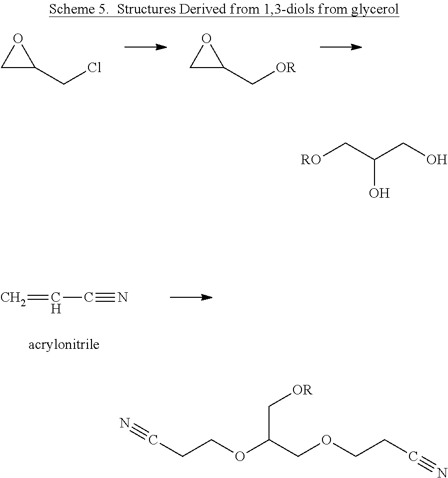

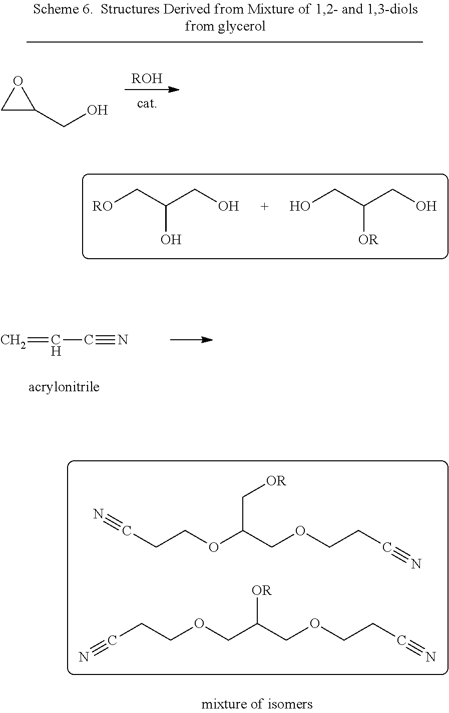

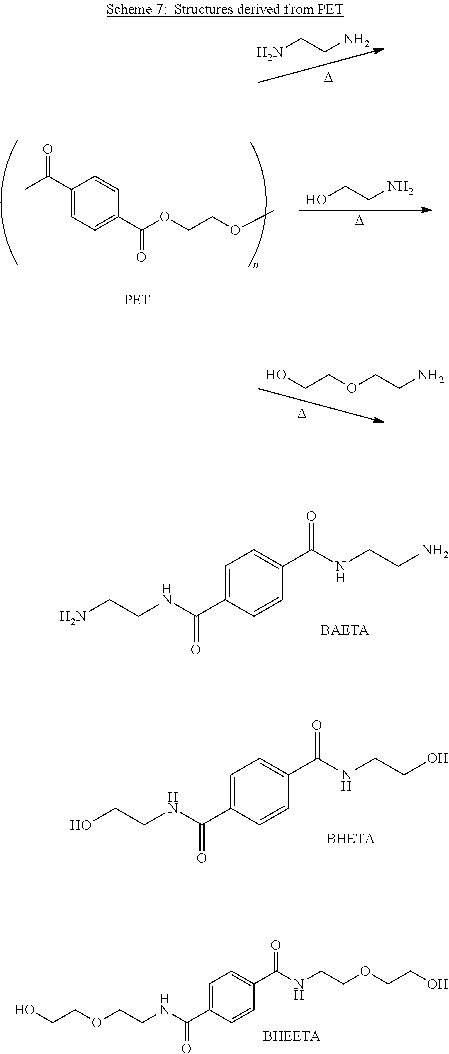

BHETA=Bis(2-hydroxyethyl)terephthalamide
BHEETA=Bis(2-(2-hydroxyethoxy)ethyl)terephthalamide
BAETA=Bis(2-aminoethyl)terephthalamide

These terephthalamides can be further modified for 3D printing, membranes, and other new polymers, resins, and elastomers.

The compositions and methods of the appended claims are not limited in scope by the specific compositions and methods described herein, which are intended as illustrations of a few aspects of the claims, and any compositions and methods that are functionally equivalent are intended to fall within the scope of the claims. Various modifications of the compositions and methods, in addition to those shown and described herein, are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A crosslinking agent of Formula I 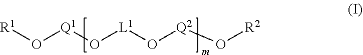
wherein:
m is 0 or 1;
Q¹ and Q², if present, are independently selected from: 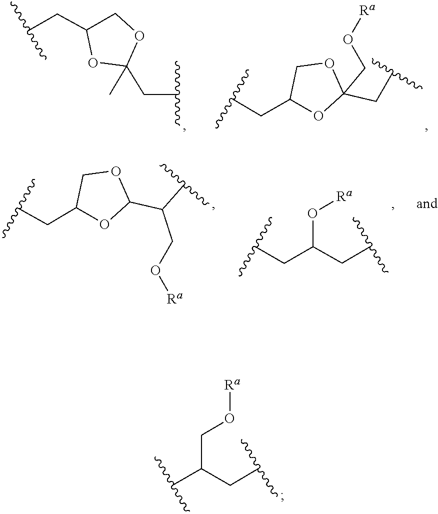
L¹, if present, is selected from C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;
R^{a} is independently selected at each occurrence from hydrogen, R¹, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;
R¹ and R² are independently selected at each occurrence from 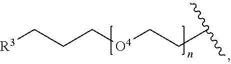
wherein n is selected at each occurrence from 0, 1, 2, or 3;
R³ is selected at each occurrence from 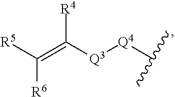
NH₂, —NH₃⁺Z⁻, and 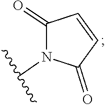 [SMILES: *N1C(=O)C=CC1=O]
Q³ is absent or independently selected at each occurrence from —C(═O)— and —S(═O)₂—;
Q⁴ is independently selected at each occurrence from —O— and —NH—;
R⁴, R⁵, and R⁶ are each independently selected at each occurrence from hydrogen, C₁-C₁₀ alkyl, C₃-Cs cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;
Z⁻ is selected at each occurrence from a halide, 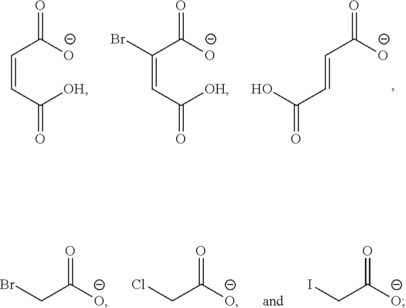
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O-(C₀-C₃ alkyl)-, R^{x}—(C₀-C₃ alkyl)-, (R^{x}R^{y}N)—(C₀-C₃ alkyl)-, R^{x}O—C(O)—(C₀-C₃ alkyl)-, R^{x}—C(O)—(C₀-C₃ alkyl)-, (R^{x}R^{y}N) C(O)—(C₀-C₃ alkyl)-, R^{x}O—S(O)₂-(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R_{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NRₓR_{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. The crosslinking agent of claim 1, wherein R¹ and R² are each

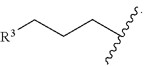

3. The crosslinking agent of claim 1, wherein R³ is selected from —NH₂, —NH₃⁺Z⁻, and

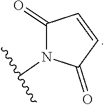 [SMILES: *N1C(=O)C=CC1=O]

4. The crosslinking agent of claim 3, wherein Z⁻ is

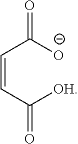

5. The crosslinking agent of claim 1, selected from:

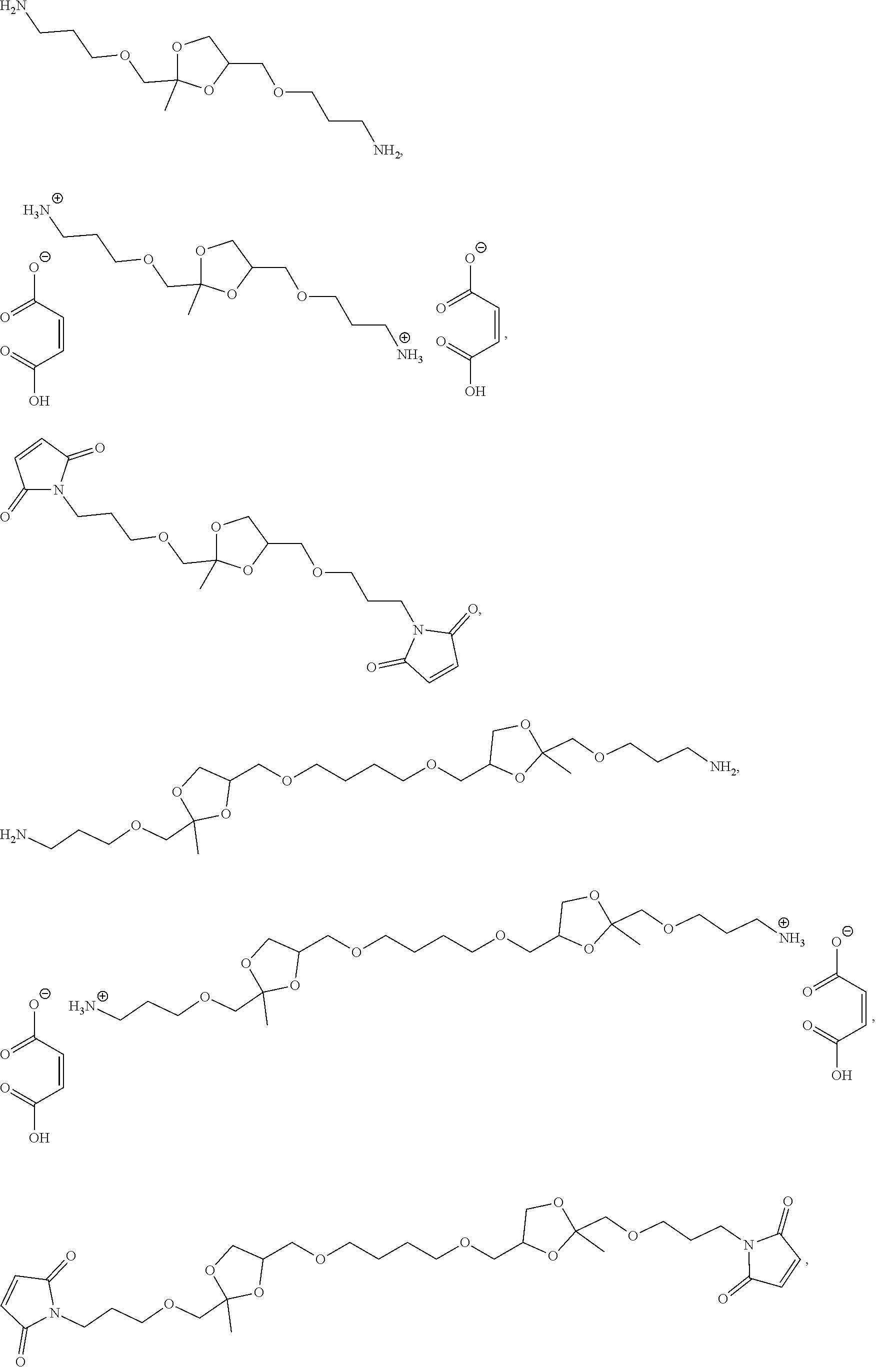

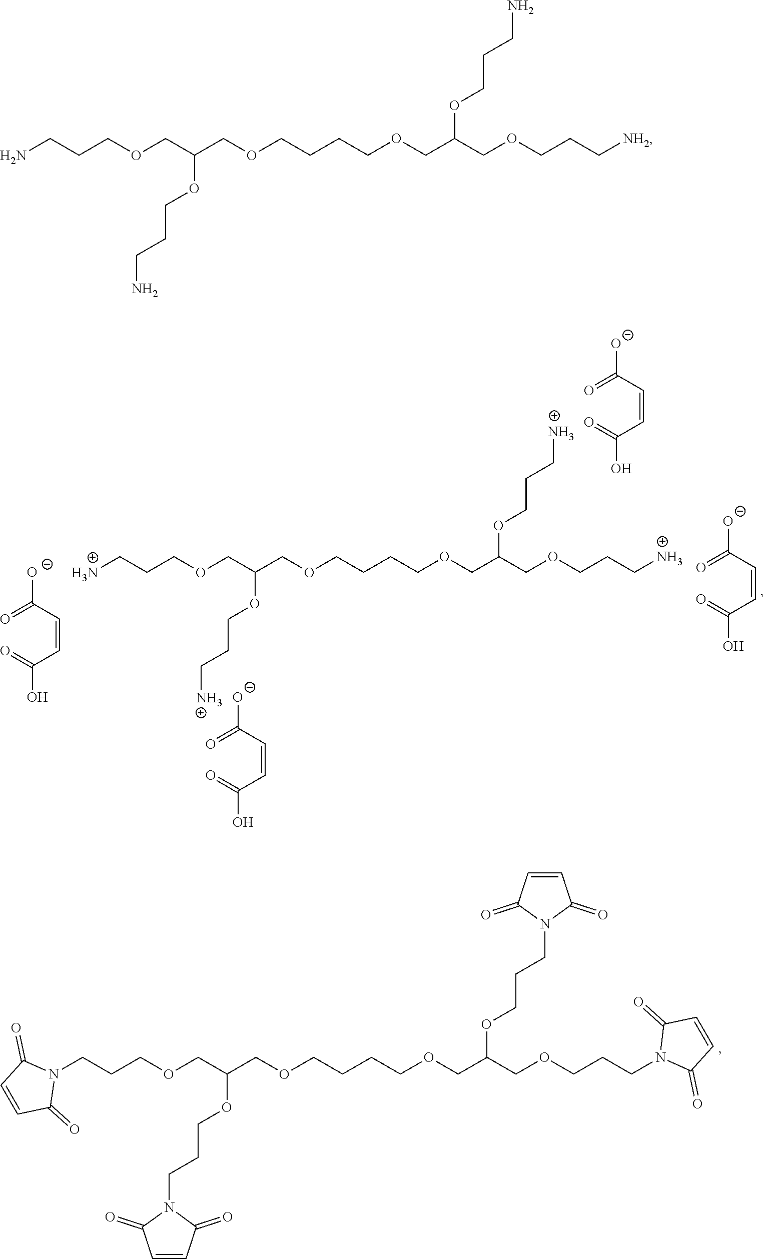

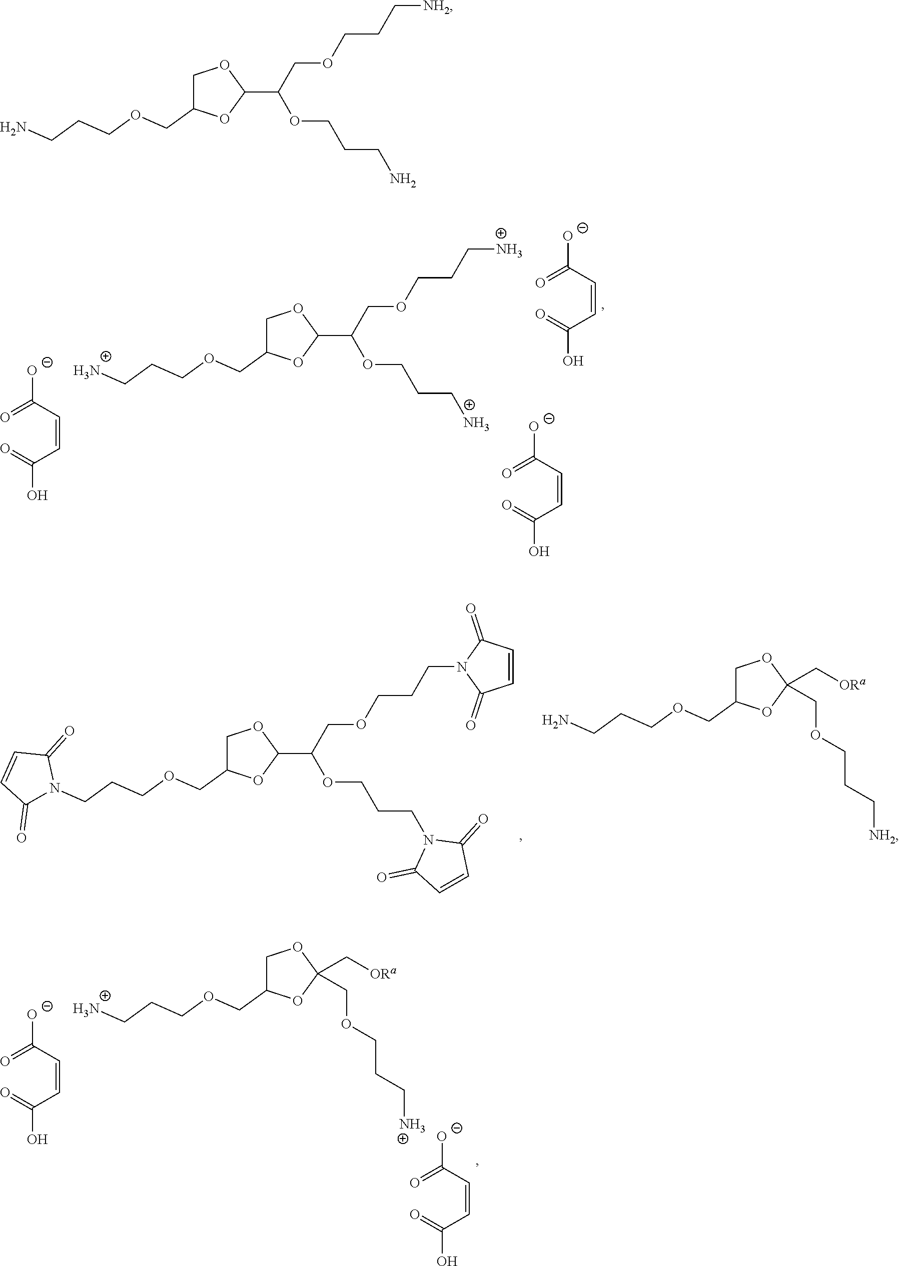

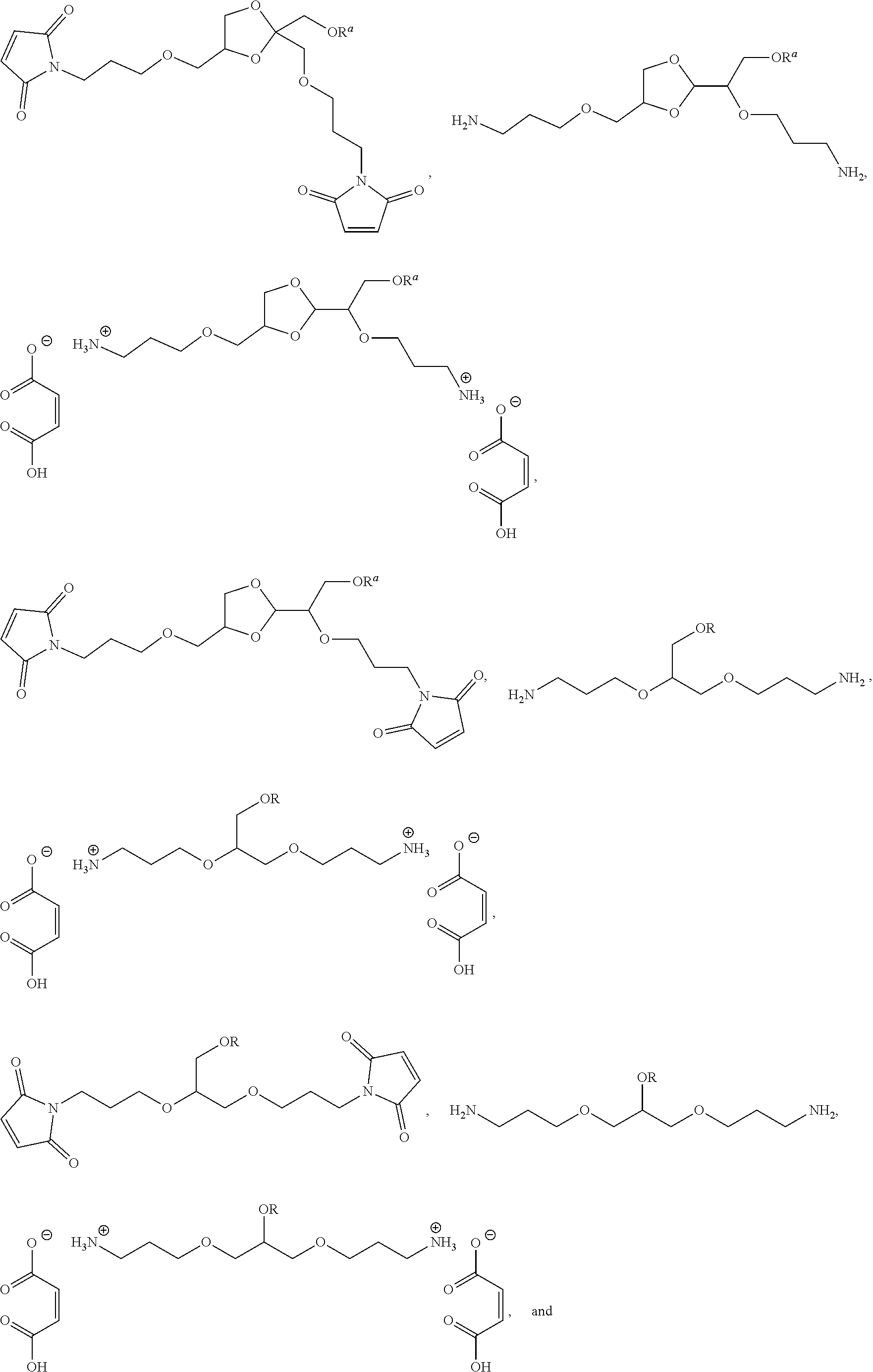

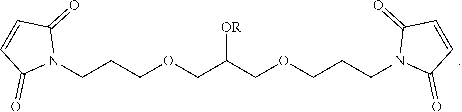

6. A polymer composition comprising at least one polymer crosslinked with a crosslinking agent of claim 1.

7. An article formed from a polymer composition of claim 6.

8. A resin composition comprising at least one monomer and a crosslinking agent of claim 1.

9. A personal care composition comprising a crosslinking agent of claim 1.

10. The personal care composition of claim 9, wherein the composition is a haircare product.

11. A crosslinking agent of Formula II 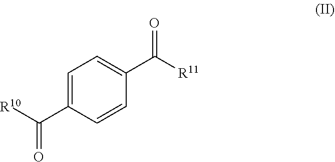
wherein:
R¹⁰ th is selected from —OR² and —NR¹R¹′;
R¹¹ is selected from —OR² and —NR²R²′;
R¹ and R² are independently selected at each occurrence from 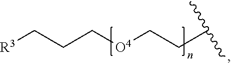
wherein n is selected at each occurrence from 0, 1, 2, or 3;
R¹′ is independently selected from hydrogen or R¹;
R²′ is independently selected from hydrogen or R²;
R³ is selected at each occurrence from 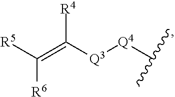
—NH₂, —NH₃⁺Z⁻, and 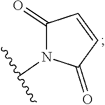 [SMILES: *N1C(=O)C=CC1=O]
Q³ is absent or independently selected at each occurrence from —C(═O)— and —S(═O)₂—;
Q⁴ is independently selected at each occurrence from —O— and —NH—;
R⁴, R⁵, and R⁶ are each independently selected at each occurrence from hydrogen, C₁-C₁₀ alkyl, C₃-C₈ cycloalkyl, 3- to 8-membered monocyclic or bicyclic heterocycle, 6- to 10-membered monocyclic or bicyclic aryl, and 5- to 10-membered monocyclic or bicyclic heteroaryl, each of which may be optionally substituted with one or more X substituents as allowed by valency;
Z⁻ is selected at each occurrence from a halide, 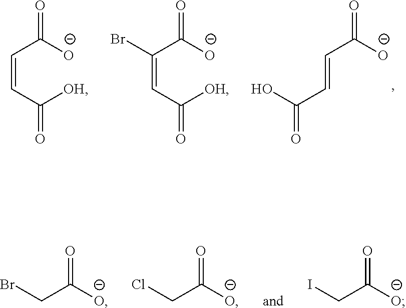
X is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O-(C₀-C₃ alkyl)-, R^{x}—(C₀-C₃ alkyl)-, (R^{x}R^{y}N)—(C₀-C₃ alkyl)-, R^{x}O—C(O)—(C₀-C₃ alkyl)-, R^{x}—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)-(C₀-C₃ alkyl)-, and R^{z}S(O)2-(C₀-C₃ alkyl)-; R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6- membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

12. The crosslinking agent of claim 11, wherein:
R¹⁰ is —NR¹R¹′;
R¹¹ is —NR²R²′;
R¹′ is hydrogen; and
R²′ is hydrogen or R².

13. The crosslinking agent of claim 11, wherein R³ is selected from —NH₂, —NH₃⁺Z⁻, and

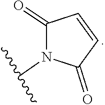 [SMILES: *N1C(=O)C=CC1=O]

14. The crosslinking agent of claim 13, wherein ⁻ is

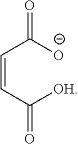

15. The crosslinking agent of claim 1, selected from:

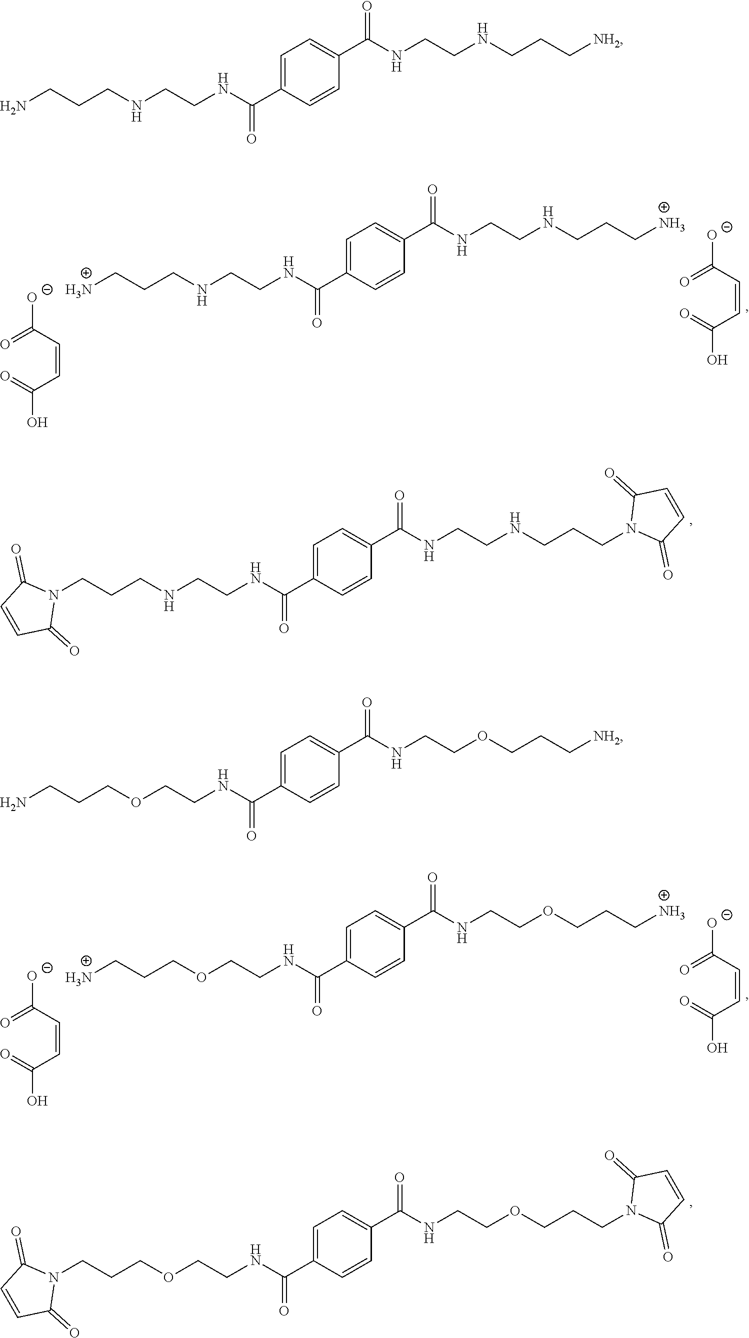

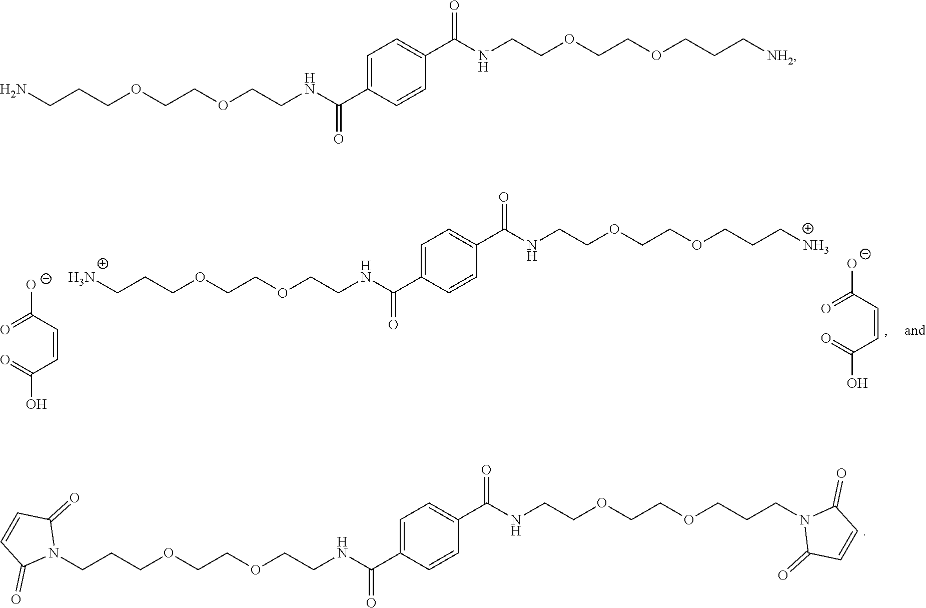 [SMILES: NCCCOCCOCCNC(=O)c1ccc(C(=O)NCCOCCOCCCN)cc1 ; O=C([O-])/C=C\C(=O)O ; [NH3+]CCCOCCOCCNC(=O)c1ccc(C(=O)NCCOCCOCCC[NH3+])cc1 ; O=C([O-])/C=C\C(=O)O ; O=C(NCCOCCOCCCN1C(=O)C=CC1=O)c1ccc(C(=O)NCCOCCOCCCN2C(=O)C=CC2=O)cc1]

16. A polymer composition comprising at least one polymer crosslinked with a cros slinking agent of claim 1.

17. An article formed from a polymer composition of claim 16.

18. A resin composition comprising at least one monomer and a crosslinking agent of claim 11.

19. A personal care composition comprising a crosslinking agent of claim 11.

20. The personal care composition of claim 19, wherein the composition is a haircare product.
